SEQUENCE LISTING (76 sequences)

SEQ ID NO: 1 - DNA, 21 bp, Artificial Sequence
  Synthetic oligonucleotide
tggcaagaaaccctaatctcc

SEQ ID NO: 2 - DNA, 20 bp, Artificial Sequence
  Synthetic oligonucleotide
ctggtctgtgaccgtcattt

SEQ ID NO: 3 - DNA, 21 bp, Artificial Sequence
  Synthetic oligonucleotide
gcccatctccatcctctattt

SEQ ID NO: 4 - DNA, 24 bp, Artificial Sequence
  Synthetic oligonucleotide
aagccataggactcttcttctatc

SEQ ID NO: 5 - DNA, 24 bp, Artificial Sequence
  Synthetic oligonucleotide
caatgctgccctttctgctccttt

SEQ ID NO: 6 - DNA, 24 bp, Artificial Sequence
  Synthetic oligonucleotide
cacttgggtgtttgagcattgcct

SEQ ID NO: 7 - DNA, 20 bp, Artificial Sequence
  Synthetic oligonucle
gaatttggtgtggacgttgg

SEQ ID NO: 8 - DNA, 23 bp, Artificial Sequence
  Synthetic oligonucleotide
cttgcactggttgacaagattag

SEQ ID NO: 9 - DNA, 22 bp, Artificial Sequence
  Synthetic oligonucleotide
accactcattgtctttgaggaa

SEQ ID NO: 10 - DNA, 21 bp, Artificial Sequence
  Synthetic oligonucleotide
attctggagggtggcaatatc

SEQ ID NO: 11 - DNA, 19 bp, Artificial Sequence
  Synthetic oligonucleotide
ggtgatgagaaggcaaaca

SEQ ID NO: 12 - DNA, 19 bp, Artificial Sequence
  Synthetic oligonucleotide
ggtgatgagaaggcaaaca

SEQ ID NO: 13 - DNA, 24 bp, Artificial Sequence
  Synthetic oligonucleotide
ttcaccaaagatctgctcctcgct

SEQ ID NO: 14 - DNA, 26 bp, Artificial Sequence
  Synthetic oligonucleotide
ttattactggtgtggagtgggtgtgg

SEQ ID NO: 15 - DNA, 24 bp, Artificial Sequence
  Synthetic oligonucleotide
cctgcattgtgaacacagcacctt

SEQ ID NO: 16 - DNA, 24 bp, Artificial Sequence
  Synthetic oligonucleotide
acctgtccactttcttctcgcagt

SEQ ID NO: 17 - DNA, 20 bp, Artificial Sequence
  Synthetic oligonucleotide
ccgggcatgaattacgagaa

SEQ ID NO: 18 - DNA, 20 bp, Artificial Sequence
  Synthetic oligonucleotide
cgaagcggtacgtgtacttt

SEQ ID NO: 19 - DNA, 22 bp, Artificial Sequence
  Synthetic oligonucleotide
cgtcacttccacgagagtttat

SEQ ID NO: 20 - DNA, 23 bp, Artificial Sequence
  Synthetic oligonucleotide
tcccacagaatgttgtagagttc

SEQ ID NO: 21 - DNA, 24 bp, Artificial Sequence
  Synthetic oligonucleotide
cgaatgacggcatctgtttgccat

SEQ ID NO: 22 - DNA, 24 bp, Artificial Sequence
  Synthetic oligonucleotide
atttggtattagggatgcagggcg

SEQ ID NO: 23 - DNA, 24 bp, Artificial Sequence
  Synthetic oligonucleotide
accaagattcccagtgaagcacct

SEQ ID NO: 24 - DNA, 24 bp, Artificial Sequence
  Synthetic oligonucleotide
tccgtctatccatgtgggcaatga

SEQ ID NO: 25 - DNA, 20 bp, Artificial Sequence
  Synthetic oligonucleotide
ctacctcgtggactgctttg

SEQ ID NO: 26 - DNA, 21 bp, Artificial Sequence
  Synthetic oligonucleotide
gttaagcaccagggagatgag

SEQ ID NO: 27 - DNA, 24 bp, Artificial Sequence
  Synthetic oligonucleotide
aatcttcctgaccaccctgcatgt

SEQ ID NO: 28 - DNA, 24 bp, Artificial Sequence
  Synthetic oligonucleotide
cagccacagccagcataaagacaa

SEQ ID NO: 29 - DNA, 20 bp, Artificial Sequence
  Synthetic oligonucleotide
gagtgagagagtcgtgtttc

SEQ ID NO: 30 - DNA, 20 bp, Artificial Sequence
  Synthetic oligonucleotide
acttctgtccctttcctttc

SEQ ID NO: 31 - DNA, 18 bp, Artificial Sequence
  Synthetic oligonucleotide
tctgcctttgtgtgtgtg

SEQ ID NO: 32 - DNA, 18 bp, Artificial Sequence
  Synthetic oligonucleotide
gtggcagcagctcattta

SEQ ID NO: 33 - DNA, 24 bp, Artificial Sequence
  Synthetic oligonucleotide
gaaggtggagctgcattggtttga

SEQ ID NO: 34 - DNA, 24 bp, Artificial Sequence
  Synthetic oligonucleotide
taaaccagctacaggacaggccaa

SEQ ID NO: 35 - DNA, 23 bp, Artificial Sequence
  Synthetic oligonucleotide
gatgagattgatgagaccacaga

SEQ ID NO: 36 - DNA, 19 bp, Artificial Sequence
  Synthetic oligonucleotide
ttcacgcaaacagcaaacc

SEQ ID NO: 37 - DNA, 24 bp, Artificial Sequence
  Synthetic oligonucleotide
tgctacaaccgtgccagtgactat

SEQ ID NO: 38 - DNA, 24 bp, Artificial Sequence
  Synthetic oligonucleotide
agtggtctttcaggtgtgagcagt

SEQ ID NO: 39 - DNA, 19 bp, Artificial Sequence
  Synthetic oligonucleotide
cacgccctttaccacatcc

SEQ ID NO: 40 - DNA, 20 bp, Artificial Sequence
  Synthetic oligonucleotide
aggttcagagagctggagaa

SEQ ID NO: 41 - DNA, 24 bp, Artificial Sequence
  Synthetic oligonucleotide
tgtccctaccttgtctgttagcca

SEQ ID NO: 42 - DNA, 24 bp, Artificial Sequence
  Synthetic oligonucleotide
attggaacatggcctctggatgga

SEQ ID NO: 43 - DNA, 24 bp, Artificial Sequence
  Synthetic oligonucleotide
cctgctgccttacacagtgcattt

SEQ ID NO: 44 - DNA, 24 bp, Artificial Sequence
  Synthetic oligonucleotide
tgcgactatgaacaagcctacggt

SEQ ID NO: 45 - DNA, 26 bp, Artificial Sequence
  Synthetic oligonucleotide
tttgcattcactcctgcggagaccta

SEQ ID NO: 46 - DNA, 24 bp, Artificial Sequence
  Synthetic oligonucleotide
actcattgcacgctgcataatcgc

SEQ ID NO: 47 - DNA, 20 bp, Artificial Sequence
  Synthetic oligonucleotide
cgggtccaccagtttgaatg

SEQ ID NO: 48 - DNA, 21 bp, Artificial Sequence
  Synthetic oligonucleotide
gttgtattggttcggcaccat

SEQ ID NO: 49 - DNA, 26 bp, Artificial Sequence
  Synthetic oligonucleotide
tttggcaactaacgtagaaactcaac

SEQ ID NO: 50 - DNA, 23 bp, Artificial Sequence
  Synthetic oligonucleotide
tgccaagagcatgaatacagaga

SEQ ID NO: 51 - DNA, 24 bp, Artificial Sequence
  Synthetic oligonucleotide
gggtctctgctgcatttgtgtcat

SEQ ID NO: 52 - DNA, 24 bp, Artificial Sequence
  Synthetic oligonucleotide
agaggcgaggaagtcaccatcaaa

SEQ ID NO: 53 - DNA, 24 bp, Artificial Sequence
  Synthetic oligonucleotide
tcctgctcttcttgcatctggctt

SEQ ID NO: 54 - DNA, 22 bp, Artificial Sequence
  Synthetic oligonucleotide
tgtaacccggacgtttccaagt

SEQ ID NO: 55 - DNA, 21 bp, Artificial Sequence
  Synthetic oligonucleotide
cgggaagattccatcagatcc

SEQ ID NO: 56 - DNA, 22 bp, Artificial Sequence
  Synthetic oligonucleotide
gtctttcaacccgtctatctcc

SEQ ID NO: 57 - DNA, 21 bp, Artificial Sequence
  Synthetic oligonucleotide
cccagaacttcgtaccctttg

SEQ ID NO: 58 - DNA, 19 bp, Artificial Sequence
  Synthetic oligonucleotide
tgcttgggaggaggtctta

SEQ ID NO: 59 - DNA, 20 bp, Artificial Sequence
  Synthetic oligonucleotide
ccgaaagccactctgtaatc

SEQ ID NO: 60 - DNA, 21 bp, Artificial Sequence
  Synthetic oligonucleotide
cctgaggaagctgttacattc

SEQ ID NO: 61 - DNA, 23 bp, Artificial Sequence
  Synthetic oligonucleotide
ccgagccttgtatgtatgttatg

SEQ ID NO: 62 - DNA, 22 bp, Artificial Sequence
  Synthetic oligonucleotide
tttgtacaccaagcacctattt

SEQ ID NO: 63 - DNA, 19 bp, Artificial Sequence
  Synthetic oligonucleotide
tcgctgctcaagtcctaca

SEQ ID NO: 64 - DNA, 23 bp, Artificial Sequence
  Synthetic oligonucleotide
ccaggagagataatctggactca

SEQ ID NO: 65 - DNA, 21 bp, Artificial Sequence
  Synthetic oligonucleotide
tggattgcctgtgaggattac

SEQ ID NO: 66 - DNA, 21 bp, Artificial Sequence
  Synthetic oligonucleotide
ttaggagcctccgtttgaatg

SEQ ID NO: 67 - DNA, 22 bp, Artificial Sequence
  Synthetic oligonucleotide
gaaagagaggaccaaccagaat

SEQ ID NO: 68 - DNA, 21 bp, Artificial Sequence
  Synthetic oligonucleotide
ttgggtacgagttgcctttag

SEQ ID NO: 69 - DNA, 24 bp, Artificial Sequence
  Synthetic oligonucleotide
tgagttgctcataccgtgctgcta

SEQ ID NO: 70 - DNA, 24 bp, Artificial Sequence
  Synthetic oligonucleotide
ccctcaaaccaacttgtcaacagc

SEQ ID NO: 71 - DNA, 22 bp, Artificial Sequence
  Synthetic oligonucleotide
gcgtctatgatggcaagatgta

SEQ ID NO: 72 - DNA, 20 bp, Artificial Sequence
  Synthetic oligonucleotide
acccaggtgataccgatagt

SEQ ID NO: 73 - DNA, 22 bp, Artificial Sequence
  Synthetic oligonucleotide
tggaaagatgaaacctcgttat

SEQ ID NO: 74 - DNA, 19 bp, Artificial Sequence
  Synthetic oligonucleotide
tcctaagcaccggatagtt

SEQ ID NO: 75 - DNA, 22 bp, Artificial Sequence
  Synthetic oligonucleotide
agcatcacgatgaccttgaata

SEQ ID NO: 76 - DNA, 21 bp, Artificial Sequence
  Synthetic oligonucleotide
ggcacttgaaagctgtttctt

(19) United States
(12) Patent Application Publication
(10) Pub. No.: US 2023/0265391 A1
(43) Pub. Date: Aug. 24, 2023

(54) METHODS AND COMPOSITIONS FOR MAKING AND USING ENDOCARDIAL CELLS

(71) Applicant: University Health Network, Toronto, Ontario (CA)

(72) Inventors: Gordon M. Keller, Toronto, Ontario (CA); Alexander Mikryukov, Toronto, Ontario (CA); Amine Mazine, Toronto, Ontario (CA); Ian Fernandes, Toronto, Ontario (CA)

(21) Appl. No.: 17/924,040

(22) PCT Filed: May 7, 2021
(86) PCT No.: PCT/IB2021/053915

§ 371 Date: Nov. 8, 2022

Related U.S. Application Data
(60) Provisional application No. 63/021,999, filed on May 8, 2020.

(51) Int. Cl. C12N 5/077; C12N 5/071

(52) U.S. Cl. CPC C12N 5/0657; C12N 5/069; C12N 2501/155; C12N 2501/415; C12N 2501/115; C12N 2501/15; C12N 2501/165; C12N 2506/02

(57) ABSTRACT

Methods and compositions for making endocardial cells from pluripotent stem cells are described, as are methods and compositions for using such cells.

### CROSS-REFERENCE TO RELATED APPLICATIONS

This application claims the benefit of U.S. Application No. 63/021,999, filed May 8, 2020. The disclosure of the prior application is considered part of (and is incorporated by reference in) the disclosure of this application.

### TECHNICAL FIELD

This disclosure relates to endocardial cells, and methods and compositions for making and using such cells.

### BACKGROUND

The heart is comprised of cardiomyocyte and non-cardiomyocyte lineages that are specified at different times from distinct progenitor populations during embryonic development. Interactions between the different cell types are essential for heart development as well as for maintaining homeostasis and normal function in the adult organ. One of the earliest stages of heart development is the formation of the primitive heart tube, which consists of an inner layer of specialized endothelial cells, known as endocardial cells, surrounded by an outer layer of cardiomyocytes. These endocardial cells play a pivotal role in heart development, as they are responsible for inducing the first functional population of cardiomyocytes, namely trabecular cardiomyocytes.

Trabecular cardiomyocytes form finger-like projections, known as trabeculae that protrude into the lumen of the developing atria and ventricles and function to rapidly increase muscle mass during embryonic life. Induction of the trabecular fate in cardiomyocytes is mediated through neuregulin/ERBB2 signaling via neuregulin secreted by the endocardial cells. These early induction steps are essential for normal heart development, as disruption of endocardial specification and formation results in the absence of trabeculation, impaired maturation and embryonic lethality. In addition to the formation of the first contracting tissue, the trabecular myocardium also gives rise to the Purkinje fibers, a subpopulation of the conduction system cells. As development proceeds, much of the trabecular myocardium is replaced by compact myocardium made up of compact cardiomyocytes. Compact myocardium forms the force-generating tissue required for heart function in the adult.

Beyond induction of trabecular myocardium, the embryonic endocardium serves as a source of progenitors for several other types of cells in the heart. Lineage tracing and gene targeting studies have shown that the embryonic endocardium gives rise to a portion of the endothelium that makes up the coronary vasculature in the heart. The coronary endothelium differs from other endothelia in that it shows unique responses to blood osmolarity and a high capacity to transport fatty acids, suited to function in an organ of high energy demands. In addition to coronary endothelium, the endocardium also gives rise to valvular endothelial cells (VECs) and valvular interstitial cells (VICs), the types of cells that form the heart valves.

In addition to its unique functional properties, the endocardial lineage is distinguished from other endothelial populations by its developmental origin. The endocardial lineage is specified from a progenitor population that expresses NKX2-5, a key cardiac transcription factor, and ISL1, a regulator of secondary heart field development. During endocardial development, NKX2-5 directly activates ETV2, a transcription factor that is essential for development of the endothelial and endocardial lineages. Within the pool of NKX2-5⁺ cells, ETV2 functions to upregulate NFATC1 expression, which, in turn, promotes endocardial development at the expense of the cardiomyocyte fate.

Given the central role of the endocardial lineage in heart development, the ability to generate these endothelial cells from pluripotent stem cells (PSCs) (e.g., human PSCs (hPSCs)) would provide a new and potentially unlimited source of these cells for generating coronary endothelial cells and biological valves for therapeutic applications, for engineering cardiac tissues for disease modeling and drug screening, and for use in studying their developmental origin.

### SUMMARY

A reporter cell line was used to identify and characterize the regulatory pathways that promote the development of a NKX2-5⁺ CD31⁺ endocardial-like population. The cells generated under these conditions express the collection of markers that define the endocardial lineage in vivo and demonstrate the capacity to induce a trabecular fate. The analyses of signaling pathways described herein identified BMP10 as a key regulator of this population. The characteristics of these NKX2-5⁺ CD31⁺ cells distinguish them from hPSC-derived NKX2-5⁻ endothelial cells generated in the absence of BMP10.

In one aspect, methods of producing a population of endocardial cells are provided. Such methods typically include providing cardiovascular progenitor cells; and contacting the cardiovascular progenitor cells with FGF and BMP under appropriate culture conditions, thereby producing a population of endocardial cells, wherein the population of endocardial cells is phenotypically NKX2-5+ and CD31+.

In some embodiments, the population of endocardial cells is phenotypically GATA4+, GATA5+, NFATC1+, NPR3+ and NRG1+. In some embodiments, the population of endocardial cells is phenotypically ENG(CD105)+.

In some embodiments, the cardiovascular progenitor cells are pluripotent stem cell-derived cardiovascular progenitor cells. In some embodiments, the cardiovascular progenitor cells are derived from cardiovascular mesoderm cells. In some embodiments, the cardiovascular progenitor cells are human cells.

In some embodiments, the BMP is selected from BMP10 and BMP4. In some embodiments, the FGF is bFGF. In some embodiments, the appropriate culture conditions comprises the absence of VEGF. In some embodiments, the appropriate culture conditions comprises the absence of a Wnt inhibitor.

In another aspect, endocardial cells made by such methods are provided.

In some embodiments, such methods further include culturing PDGFRb+ cells from the population of endocardial cells in the presence of bFGF, BMP2 (or BMP4) and TGFbeta2, thereby producing valvular interstitial-like cells (VICs). In some embodiments, VICs are phenotypically SOX9+, MSX2+, VIM+, VCAN+, COLIA1+, COL3A1+, POSTN+, CDH11+, NR4A2+, PRRX2+, TIMP3+, RGS5+ and ITGA2+. In still another aspect, valvular interstitial-like cells (VICs) made by such methods are provided.

In another aspect, methods of producing coronary endothelial-like cells are provided. Such methods typically include contacting the endocardial cells described herein with VEGFA followed by VEGFB, thereby producing coronary endothelial-like cells.

In some embodiments, such methods further include culturing the population of endocardial cells in the presence of ventricular cardiomyocytes, wherein the ventricular cardiomyoctyes are phenotypically MYL2(MLC2V)+ and CTNT+, thereby generating trabecular myocardial cells, wherein the trabecular myocardial cells are phenotypically BMP10+, NPPA+, NPPB+, IRX3+, GJA5+, MYL2+, and CTNT+. In some embodiments, the culturing is in the presence of VEGFA.

In yet another aspect, trabecular ventricular cardiomyocytes made by such methods are provided, wherein the trabecular ventricular cardiomyocytes are phenotypically BMP10+, NPPA+, NPPB+, IRX3+, GJA5+, MYL2+, and CTNT+.

In one aspect, methods of replenishing coronary vasculature in myocardium are provided. Such methods typically include delivering the endocardial cells described herein and/or the coronary endothelial cells described herein to heart tissue, thereby replenishing the coronary vasculature. In some embodiments, the heart tissue is damaged.

In another aspect, methods of improving cardiomyocyte grafts by replenishing the myocardium are provided. Such methods typically include delivering ventricular cardiomyocytes in conjunction with the endocardial cells described herein and/or the coronary endothelial cells described herein, thereby replenishing the myocardium. In some embodiments, the heart tissue is damaged.

In still another aspect, methods of screening for test compounds that exhibit toxicity to endocardial cells, valvular interstitial-like cells or trabecular cardiomyocytes are provided. Such methods typically include contacting the endocardial cells described herein, the valvular interstitial-like cells described herein, the coronary endothelial-like cells described herein and/or the trabecular ventricular cardiomyocytes described herein with a test compound, and identifying test compounds that reduce the viability of any of such cells. In some embodiments, the test compounds can be proteins, small molecules, and nucleic acids.

In still another aspect, methods of producing valvular interstitial-like cells are provided. Such methods typically include contacting the endocardial cells described herein with BMP2/4 and TGFbeta2 under appropriate culture conditions, or contacting the endocardial cells described herein with a) CHIR99021, SB-431542, bFGF and BMP2/4 followed by b) bFGF, BMP2/4 and TGFbeta2, thereby producing valvular interstitial-like cells. In some embodiments, the valvular interstitial-like cells (VICs) are phenotypically SOX9+, MSX2+, VIM+, VCAN+, COLIA1+, COL3A1+, POSTN+, CDH11+, NR4A2+, PRRX2+, TIMP3+, RGS5+ and ITGA2+.

In yet another aspect, methods of producing trabecular ventricular cardiomyocytes are provided. Such methods typically include contacting ventricular cardiomyocytes with the endocardial cells described herein or exogenous neuregulin (NRG1) under appropriate culture conditions, thereby producing trabecular ventricular cardiomyocytes. In some embodiments, the exogenous NRG1 is recombinant.

In another aspect, methods of inducing the production of neuregulin in a cell are provided. Such methods typically include contacting the endocardial cells described herein with ventricular cardiomyocytes under appropriate conditions.

In yet another aspect, biological valves made using the endocardial cells described herein and/or the valvular interstitial-like cells described herein are provided.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1I shows data from experiments showing generation of NKX2-5+ CD31+ cells from cardiogenic mesoderm. FIG. 1A is a schematic of the strategy used for identifying the key pathways that regulate the generation of NKX2-5+ CD31+ cells from hPSCs. FIG. 1B shows representative flow cytometric analyses of effects of bFGF (concentrations in ng/ml indicated on the graph), BMP4 (10 ng/ml) and BMP10 (10 ng/ml) on the generation of NKX2-5+ and CD31+ populations at day 9 of differentiation. FIG. 1C-1E show the quantification of frequency of NKX2-5+ CD31+ cells generated following BMP specification from: day5-day9 (D5-D9) (FIG. 1C), day3-day9 (D3-D9) (FIG. 1D) and day7-day9 (D7-D9) (FIG. 1E). Comparisons are made with the optimal condition indicated by an arrow (n=4, ANOVA *P<0.05, **P<0.005, ***P<0.0005). FIG. 1F shows the total number of NKX2-5+ CD31+ cells (per well) generated following BMP (10 ng/ml) specification for the indicated time in the presence of bFGF (50 ng/ml). FIG. 1G-1I shows the titration of BMP4 and BMP10 for generation of NKX2-5+ CD31+ cells in FIG. 1A. FIG. 1G shows the flow cytometric analyses of the proportion of NKX2-5+ CD31+ cells generated in day 9 populations specified from days 5-9 with the indicated concentrations (ng/ml) of either BMP4 or BMP10 in the presence of bFGF (50 ng/ml). FIG. 1H, 1I show the quantification of the frequency and total number of NKX2-5+ CD31+ cells (per well) generated at the indicated concentrations of BMP4 or BMP10 as detailed in FIG. 1G. Comparisons are made with the optimal condition indicated by the arrow. Data are represented as mean±SEM (n=4-10, ANOVA *P<0.05, **P<0.005, ***P<0.0005).

FIG. 2A-2E is data showing the kinetics of induction of NKX2-5+ CD31+ cell population. FIG. 2A is a schematic of the protocol used to define the kinetics of the development of the NKX2-5+ CD31+ and NKX2-5− CD31+ populations. Time points analyzed are indicated by the arrows. FIG. 2B are representative flow cytometric analyses showing the development of NKX2-5+ CD31+ and NKX2-5− CD31+ cells at the indicated times of differentiation. FIG. 2C-2E are graphs showing the quantification of frequency, the total number of cells and the total number of NKX2-5+ CD31+ generated at the indicated times under the indicated conditions. Data are represented as mean±SEM (n=5-10, ANOVA *P<0.05, **P<0.005, ***P<0.0005).

FIG. 3A-3E is experimental data showing characterization of the NKX2-5+ CD31+ endocardial-like cells. FIG. 3A is a schematic of the protocol used for the generation and isolation of hESC-derived NKX2-5+ CD31+ endocardial-like cells and NKX2-5− CD31+ control endothelial cells. The gating strategy used for the isolation of different NKX2-5 and CD31 populations is indicated in different colors. FIG. 3B are graphs of RT-qPCR analysis of the expression levels of endocardium-specific genes (NFATC1, NRG1, NPR3), endocardial progenitor-specific genes (NKX2-5, ETV2, ISL1), cardiac transcription program-specific genes (GATA4, GATA5) and components of NOTCH signaling pathway (DLL4, NOTCH1, JAG1, NOTCH2) in the indicated isolated day 9 populations: SIRPA+ cardiomyocytes (red), NKX2-5− CD31+ control endothelial cells (blue), BMP10-induced NKX2-5− CD31 cells (black), NKX2-5+ CD31 cells (orange), NKX2-5+ CD31+ cells (green) and NKX2-5 CD31+ (grey) cells (n=7, paired t-test *P<0.05, **P<0.01, ***P<0.005). FIG. 3C are graphs of RT-qPCR analysis of the expression levels of cardiac transcription program-specific genes (GATA4, GATA5) and endocardium-specific genes (NFATC1, NRG1) in NKX2-5+ CD31+ populations specified with either BMP10 (E-B10) or BMP4 (E-B4) and in NKX2-5− CD31+ control endothelial cells (n=5, paired t-test *P<0.05, **P<0.01, ***P<0.005). FIG. 3D shows flow cytometric analyses of NKX2-5 expression in day 9 BMP10-induced NKX2-5+ CD31+ populations cultured for 8 days in the presence of VEGFA (100 ng/ml) and the indicated concentration of BMP4 or BMP10. FIG. 3E is a graph of RT-qPCR analyses of NRG1 expression in day 9 NKX2-5+ CD31+ [E-B10 (D9 a.s)] and control NKX2-5− CD31+ [Cnt-E (D9 a.s)] immediately after FACS and following culture for 8 days with VEGFA (100 ng/ml) in absence of BMP or in the presence of BMP10 (10 ng/ml) or BMP4 (10 ng/ml) (n=3, paired t-test *P<0.05, **P<0.01, ***P<0.005). For all PCR analyses, expression values were normalized to the housekeeping gene TBP. Error bars represent SEM. CMs, cardiomyocytes; D, day; a.s., after sort; E-B4, BMP4 induced endocardium; E-B10, BMP10 induced endocardium; Ctrl-E, control endothelium.

FIG. 4A-4E is data showing that neuregulin signaling specifies a trabecular fate in cardiomyocytes. FIG. 4A is a schematic of the strategy used for identifying the NRG1 responsive cardiomyocyte population. SIRPA+ cells were isolated at the indicated times by FACS and cultured for 8 days as aggregates in the presence of NRG1. FIG. 4B shows RT-qPCR analysis of the expression levels of trabecular myocardium-specific genes (BMP10, NPPA, NPPB, IRX3, GJA5), a compact myocardium-specific gene (HEY2), a ventricular cardiomyocyte-specific gene (MYL2) and a pan-cardiomyocyte gene (CTNT) in SIRPA+ cells in the indicated populations cultured in the presence or absence of NRG1 (50 ng/ml) (n=5, ANOVA *P<0.05, **P<0.005, ***P<0.0005). FIGS. 4C and 4D show representative flow cytometric analyses of the proportion of MLC2V+/ANP+ (4C) and MLC2V+/CTNT+ (4D) cells in aggregates of SIRPA+ cardiomyocytes isolated at the indicated times and cultured 8 days with or without NRG1. FIG. 4E is a graph showing the quantification of the percentage of ANP+ MLC2V+ and ANP-MLC2V+ cells in aggregates analyzed in (4C) and (4D). The frequencies of ANP+ or ANP− cells in D16 and D23 aggregates are compared to the frequencies of ANP+ or ANP− cells in D9 aggregates (n=5, paired t-test *P<0.05, **P<0.01, ***P<0.005). For all PCR analyses, expression values were normalized to the housekeeping gene TBP. Error bars in all graphs represent SEM. CMs, cardiomyocytes; D, day; a.s., after sort; N, NRG1.

FIG. 5A-5F is data showing the co-culture of NKX2-5+ CD31+ endocardial-like cells and SIRPA+ cardiomyocytes. FIG. 5A is a schematic of the experimental strategy used to test the trabecular inductive effects of the NKX2-5+ CD31+ cells and the control endothelial cells on the SIPRA+ target cardiomyocyte population. RFP+ SIRPA+ cardiomyocytes and NKX2-5+ CD31+ endocardial-like cells or NKX2-5− CD31+ control endothelial cells were isolated at day 9 of differentiation, mixed and plated in a monolayer format and cultured for 8 days in the presence of VEGFA (100 ng/ml) with or without ERBB2 inhibitor lapatinib (lap, 10 μM). FIG. 5B are photomicrographs of the co-cultures showing the segregation of the cardiomyocyte aggregates on the endothelial cell monolayer. Merge of the bright field image and the RFP channel. Scale bars represent 200 μm. FIG. 5C are flow cytometry analyses of populations following co-culture. The populations were segregated into RFP+ and RFP− fractions and then further segregated as indicated. FIG. 5D are graphs of RT-qPCR analysis showing the expression levels of trabecular myocardium-specific genes (BMP10, NPPA, NPPB, IRX3, GJA5), a compact myocardium-specific gene (HEY2), a ventricular cardiomyocyte-specific gene (MYL2) and a pan-cardiomyocyte gene (CTNT) in RFP+ SIRPA+ cells. For statistical analysis, expression levels in all groups were compared to cardiomyocytes cultured with the NKX2-5+ CD31+ endocardial-like cells (indicated with an arrow) (n=5-10, paired t-test *P<0.05, **P<0.01, ***P<0.005). FIG. 5E are representative flow cytometric analyses of NKX2-5/CD31 expression in the indicated populations cultured for 8 days in the presence of different concentrations of the pan-BMP inhibitor LDN193189. FIG. 5F shows RT-qPCR analysis of the expression levels of trabecular myocardium-specific genes (BMP10, NPPA, IRX3) and a compact myocardium-specific gene (HEY2) in RFP+SIRPA+ cells cultured (8 days) with either BMP10- or BMP4-specified NKX2-5+ CD31+ cells or with control NKX2-5− CD31+ cells (n=7, paired t-test *P<0.05, **P<0.01, ***P<0.005). For all PCR analyses, expression values were normalized to the housekeeping gene, TBP. Error bars in all graphs represent SEM. CMs, cardiomyocytes; aggs, aggregates; D, day; a.s., after sort; E-B4, BMP4 induced endocardium; E-B10, BMP10 induced endocardium; Ctrl-E, control endothelium; lap, lapatinib.

FIG. 6A-6F is data showing the generation of endocardial-like cells from a HES2-RFP line. FIG. 6A is a schematic of the differentiation protocol used for generating endocardial-like and control endothelial cells from HES2-RFP hPSC cells. FIG. 6B are heat maps showing the average frequency of CD31+ endocardial-like cells generated at day 9 of differentiation from the mesoderm populations induced with the indicated concentrations of BMP4 and activin A. The frequency of the CD31+ cells generated from the different populations was compared to the frequency generated by the 5B/4A induced mesoderm (n=4, ANOVA *P<0.05, **P<0.005, ***P<0.0005). FIG. 6C is a schematic of the experimental strategy used for co-culture of the HES2-RFP hPSC-derived endothelial cells and cardiomyocytes. GFP+ SIRPA+ cardiomyocytes and RFP+ CD31+ endocardial-like (Endo) or control endothelial cells (Ctrl-E) were isolated at day 9 of differentiation, mixed and cultured in a monolayer format for 8 days in the presence of VEGFA (100 ng/ml) with or without ERBB2 inhibitor lapatinib (lap, 10 μM). Following co-culture, the populations were isolated and analyzed. FIG. 6D shows flow cytometry analyses of populations following co-culture. Fractions were isolated as indicated. FIG. 6E are graphs showing RT-qPCR analysis of the expression levels of trabecular myocardium-specific genes (BMP10, NPPA, NPPB, IRX3, GJA5), a compact myocardium-specific gene (HEY2), a ventricular cardiomyocyte-specific gene (MYL2) and a pan-cardiomyocyte gene (CTNT) in the isolated GFP+SIRPA+ cardiomyocytes following co-culture. For the statistical analysis, expression levels in all groups were compared to those in the cardiomyocytes cultured with the BMP10-induced cells (CMs+Endo) (n=7-9, paired t-test *P<0.05, **P<0.01, ***P<0.005). FIG. 6F are graphs showing RT-qPCR analysis of the expression levels of NRG1 and NKX2-5 in RFP+ CD31+ endocardial-like and control endothelial cells in isolated populations prior to and following 8 days of co-culture (CC) (n=9, paired t-test *P<0.05, **P<0.01, ***P<0.005). For all PCR analyses, expression values were normalized to the housekeeping gene, TBP. Error bars in all graphs represent SEM. CMs, cardiomyocytes; aggs, aggregates; D, day; a.s., after sort; Endo, BMP10 induced endocardium; Ctrl-E, control endothelium; lap, lapatinib.

FIG. 7 is a schematic of a model proposed herein. A model summarizing the NRG1- and BMP10-mediated interactions between the cardiomyocytes and endocardial cells in the hPSC-derived cultures and the developing heart in vivo is shown. In this model, NRG1 secreted by the endocardial cells induces a trabecular fate, including BMP10 expression in the cardiomyocytes. The cardiomyocyte-derived BMP10 acts on endocardial cells, maintaining their expression of NKX2-5 and NRG1.

FIG. 8A-8J is data showing the effects of WNT and VEGF signaling on generation of NKX2-5+ CD31+ cells from cardiogenic mesoderm. FIG. 8A is a schematic of the experimental strategy used to analyze the effects of Wnt signaling on the generation of NKX2-5+ CD31+ cells. FIG. 8B shows representative flow cytometric analyses of NKX2-5 and CD31 expression on day 9 of differentiation following inhibition (with 2 μM IWP2) or activation (with 1 μM CHIR99021) of the Wnt pathway for the indicated times. FIG. 8C-8E are graphs showing the quantification of the frequency of NKX2-5+ CD31+ cells (FIG. 8C), total cell numbers (FIG. 8D) and total number of NKX2-5+ CD31+ cells (FIG. 8E) at day 9 following the indicated treatments. Comparisons are made with the non-treated population indicated by the arrow (n=6 for frequencies, n=4 for cell counts, ANOVA *P<0.05, **P<0.005, ***P<0.0005). FIG. 8F is a schematic of the experimental strategy used to analyze the effects VEGF/KDR signaling on the generation of NKX2-5+ CD31+ cells. FIG. 8G shows representative flow cytometric analyses of NKX2-5 and CD31 expression on day 9 of differentiation following manipulation of VEGF/KDR signaling for the indicated times. FIG. 8H-8J are graphs showing the quantification of the frequency of NKX2-5+ CD31+ cells (FIG. 8H), total cell numbers (FIG. 8I) and number of NKX2-5+ CD31+ cells (FIG. 8J) at day 9 following the indicated treatments. Comparisons are made with the non-treated population indicated by the arrow (n=7 for frequencies, n=4 for cell counts, ANOVA *P<0.05, **P<0.005, ***P<0.0005).

FIG. 9A-9D is data showing expression of CD105 and cardiac transcription factors in NKX2-5+ CD31+ endocardial-like cells. FIG. 9A shows representative flow cytometric analyses of Endoglin (CD105) expression on CD31+ cells specified as indicated. FIGS. 9B-9D are photomicrographs showing immunostaining of (FIG. 9B) NKX2-5, (FIG. 9C) GATA4 and (FIG. 9D) NFATC1 (red) in BMP-10-induced endocardial cells, control endothelial cells and cardiomyocytes generated from HES3-NKX2-5eGFP/w hPSCs. The cells were co-stained with CD31 (grey) to identify endothelial cells, CTNT (grey) to identify cardiomyocytes and DAPI (blue) to visualize all cells and nuclei. The NKX2-5 immunostaining of cardiomyocytes was recorded with a lower laser intensity than for endocardial/endothelial cells as NKX2-5 expression in cardiomyocytes is significantly higher than in endocardium in correlation with RT-qPCR data. The NKX2-5 immunostaining of cardiomyocytes served as a positive control for the anti-NKX2-5 antibody. Recording parameters for the NKX2-5 immunostainings in endocardial and control endothelial cells, as well as for any other immunostainings were identical between the samples. Scale bars represent 50 μm.

FIG. 10A-10B shows the analyses of populations isolated from co-culture of NKX2-5+ CD31+ endocardial-like cells and SIRPA+ cardiomyocytes. FIG. 10A, 10B shows the analysis of various cell populations from co-cultures of endocardial-like cells and control endothelial cells with cardiomyocytes from FIG. 5B. FIG. 10A shows the flow cytometry analyses of the indicated fractions isolated from co-culture populations described in FIG. 5B. FIG. 10B are graphs showing RT-qPCR analysis of the expression levels of the trabecular myocardium-specific gene (BMP10), the pan-cardiomyocyte gene (CTNT) and the endocardium-specific (NRG1) in RFP-NKX2-5+/CD31+ endocardial/endothelial cells, RFP+ PDGFRB− SIRPA+ cardiomyocytes, RFP+ PDGFRB+ SIRPA− mesenchymal-like cells, RFP+ PDGFRB− SIRPA− CD31− cardiomyocytes and RFP+ PDGFRB− SIRPA− CD31+ endothelial cells isolated from day 8 co-cultures. For statistical analysis, endothelial populations from co-cultures of cardiomyocytes with endocardium were compared to endothelial populations from co-cultures of cardiomyocytes with control endothelial cells (n=5-10, paired t-test *P<0.05, **P<0.01, ***P<0.005). CMs, cardiomyocytes; E-B10, BMP10 induced endocardium; Ctrl-E, control endothelium; R, RFP; S, SIRPA; P, PDGFRB.

FIG. 11A-11E is experimental data showing the generation of endocardial cells from different mesoderm populations. FIG. 11A is a schematic of the protocol used for generating different mesoderm populations and derivative endocardial and control endothelial cells. FIG. 11B are representative flow cytometric analyses of CD56 and PDGFRA expression on day 3 mesoderm populations induced with the indicated amounts of BMP4 and activin A. FIG. 11C, 11F are representative flow cytometric analyses of NKX2-5 and CD31 expression on day 9 endocardial (FIG. 11C) and control endothelial (FIG. 11F) cells generated from the different mesoderm populations. FIG. 11D, 11E are heat maps showing the average frequency of NKX2-5+ CD31+ (FIG. 11D) and total CD31+ (FIG. 11E) cells generated from the different mesoderm populations. Frequencies were compared to populations generated from mesoderm induced with 10B/6A (n=4, ANOVA *P<0.05, **P<0.005, ***P<0.0005). FIG. 11G are heat maps showing the average frequency of control NKX2-5− CD31+ cells generated from the different mesoderm populations. FIG. 11H are graphs showing RT-qPCR analysis of the expression levels of trabecular myocardium-specific genes (BMP10, NPPA, IRX3) and a compact myocardium-specific gene (HEY 2) in isolated RFP+ SIRPA+ cells co-cultured with NKX2-5+ CD31+ cells generated from 3B/6A-, 5B/9A-, 10B/12A- and 10B/6A-induced mesoderm populations. For statistical analyses, all populations were compared to cardiomyocytes co-cultured with control endothelial cells generated from 10B/6A mesoderm (indicated with the arrow) (n=5, paired t-test *P<0.05, **P<0.01, ***P<0.005). FIG. 11I is a graph showing RT-qPCR analysis of the expression level of NRG1 in NKX2-5+ CD31+ endocardial cells generated from 3B/6A, 5B/9A, 10B/12A and 10B/6A mesoderm populations prior to and following (CC) co-culture with cardiomyocytes. Comparisons are made to the control endothelial cells generated from 10B/6A mesoderm (indicated with the arrow) (n=5, paired t-test *P<0.05, **P<0.01, ***P<0.005).

FIG. 12 are graphs showing gene expression profiles of CD31+ endocardial-like and control endothelial cells generated from HES2-RFP line. Graphs show RT-qPCR analysis of the expression level of NRG1,NKX2-5, GATA4, GATA5,NFATC1 and NPR3 in day 9 RFP+ CD31+ endocardial and control endothelial cells generated from mesoderm induced with 5B/4A. (n=9, paired t-test *P<0.05, **P<0.01, ***P<0.005).

FIG. 13A-13F is experimental data showing the generation and characterization of valvular intersitial-like cells (VICs) from hESC-derived endocardial-like cells. FIG. 13A is a schematic of the protocol used to generate and isolate hESC-derived CD31− PDGFRb+ mesenchymal cells from CD31+, CD31+ PDGFRb+ and CD31+ PDGFRb-endocardial-like cells and from CD31+ control endothelial cells. FIG. 13B shows representative flow cytometric analyses of endocardial and control endothelial populations before and after CD34-specific MACS beads sort as well as sub-fractioning of CD31+ CD34+ endocardial cells into PDGFRb+ and PDGFRb− populations by FACS. The bottom panel shows representative flow cytometric analyses of the effects of 8 days of treatment with bFGF (10 ng/ml), BMP2/4 (100 ng/ml) and TGFbeta2 (0.3 ng/ml) on the generation of CD31− PDGFRb+ mesenchymal cells from CD31+, CD31+ PDGFRb+, CD31+ PDGFRb− endocardial and CD31+ control endothelial cells. The gating strategy for the isolation of CD31−PDGFRb+ cells for further analysis is indicated in red. FIG. 13C-13E show the quantification of frequency of CD31− PDGFRb+ cells (13C), number of CD31− PDGFRb+ cells (13E) and total cell numbers (13D) derived from the cell populations illustrated in (13B) (n=4, paired t-test *P<0.05, **P<0.01, ***P<0.005). FIG. 13F are graphs of RT-qPCR analysis showing the expression levels of general mesenchymal genes (SOX9, VIM, VCAN, COL1A1, POSTN) and VIC-specific genes (PRRX2, NR4A2, TIMP3) in the CD31−PDGFRb+ cells illustrated in FIG. 13B as well as in the starting populations (i.e. CD31+, CD31+ PDGFRb+, CD31+ PDGFRb− endocardial cells and CD31+ control endothelial cells). For all PCR analyses, expression values were normalized to the housekeeping gene TBP. Error bars represent SEM. D, day; E 31+, CD31+ endocardial cells; E 31+ Pbeta+, CD31+ PDGFRbeta+ endocardial cells; E 31+ Pbeta−, CD31+ PDGFRbeta− endocardial cells; Ctrl 31+, CD31+ control endothelial cells.

FIG. 14A-14F is experimental data showing the method of enrichment of valvular interstitial-like cells (VICs) described in FIG. 13. FIG. 14A is a schematic of the modification of the protocol in FIG. 13A used to generate and isolate hESC-derived CD31− PDGFRb+ mesenchymal cells from CD31+ endocardial-like cells, including a 4-day expansion phase [bFGF (10 ng/ml), BMP2/4 (100 ng/ml), CHIR99021 (1 μM) and SB-431542 (5.4 μM)] followed by an 8-day VIC specification phase [bFGF (10 ng/ml), BMP2/4 (100 ng/ml) and TGFbeta2 (0.3 ng/m1)]. FIG. 14B shows representative flow cytometric analyses of VIC-like cells derived from endocardial population with protocols in FIG. 13A (T) and 14A (CH-SB/T). The gating strategy for the isolation of CD31− PDGFRb+ cells for further analysis is indicated in red. FIG. 14C-14E show the quantification of frequency of CD31− PDGFRb+cells (14C), number of CD31− PDGFRb+ cells (14E) and total cell numbers (14D) derived from the cell populations illustrated in (14B) (n=7, paired t-test *P<0.05, **P<0.01, ***P<0.005). FIG. 14F are graphs of RT-qPCR analyses showing the expression levels of general mesenchymal genes (SOX9, MSX2, VCAN, CDH11, COL1A1, COL3A1, POSTN) and VIC-specific genes (NR4A2, PRRX2, TIMP3, RGS5, ITGA2) in the CD31− PDGFRb+ cells illustrated in FIG. 14B as well as in the starting populations (i.e. CD31+ endocardial cells). For all PCR analyses, expression values were normalized to the housekeeping gene TBP. Error bars represent SEM. D, day; E 31+, CD31+ endocardial cells; CH-SB/T, VIC-like cells specified with the protocol in FIG. 14A; T, VIC-like cells specified with the protocol in FIG. 13A.

FIG. 15A-15F is experimental data showing the generation of coronary endothelial cells from endocardial cells. FIG. 15A is a schematic of the protocol for coronary endothelial cell differentiation from hPSC-derived endocardial cells. FIG. 15B shows representative flow cytometric analyses of CD34, NKX2-5, CD140b, LDLR and CD36 expression on day 9 in the endocardial populations. FIG. 15C shows representative flow cytometric analyses of CD34, NKX2-5, CD140b, LDLR and CD36 expression on day 13 in the population treated with VEGFA (50 ng) for 4 days. FIG. 15D shows representative flow cytometric analyses of LDLR and CD36 expression on the day 17 population treated with either VEGFA (50 ng) or VEGFB (50 ng) and GW7647 (1 μM) for 4 days (day 13 to 17). FIG. 15E shows RT-qPCR expression analyses of FA metabolism genes (FABP4, CD36, APOD) in day 17 endothelial cells cultured in the indicated conditions (n=4 biological replicates for VEGFA and VEGFB+PPARa treated cells) and day 9 control endothelium (CRTL-E) and endocardial cells (E-34+). Primary human coronary arterial (HCAEC) and microvascular endothelial cells (HCMEC) were included as a reference for in vivo expression. FIG. 15F shows RT-qPCR expression analyses of endocardial genes (NPR3, GATA5, GATA4) and the arterial marker (GJA4) in day 17 endothelial cells cultured in the indicated conditions (n=4 biological replicates for VEGFA and VEGFB+PPARa treated cells) and day 9 control endothelium (CRTL-E) and endocardial cells (E-34+). Primary human coronary arterial (HCAEC) and microvascular endothelial cells (HCMEC) were included as a reference for in vivo expression.

### DETAILED DESCRIPTION

The embryonic endocardium is essential for early heart development. The embryonic endocardium is, together with the myocardium, one of the first heart tissues to be formed. It is the source of the cells that make up the valves and a portion of the coronary vasculature, and it functions to induce trabecular myocardium. Given such potential, endocardial cells provide unique therapeutic opportunities that include engineering biological valves and cell-based therapy strategies to replace heart valves and coronary vasculature in damaged hearts. This disclosure describes methods of making a population of endocardial cells, as well as a number of methods for using such cells.

### Endocardial Cells

To produce a population of endocardial cells, cardiovascular progenitor cells can be cultured as described herein to generate a population of cells that displays characteristics of endocardium including expression of a cohort of genes that identifies the endocardial lineage in vivo (e.g., NKX2-5, GATA4, GATA5,NFATC1, NPR3, NRG1 and ENG(CD105)). In addition, the population of endocardial cells produced using the methods described herein have the capacity to induce a trabecular fate in immature cardiomyocytes in vitro and, surprisingly, also have the potential to produce mesenchymal cells that express markers of valve interstitial cells (VICs).

As used herein, cardiovascular progenitor cells can refer to pluripotent stem cell (PSC)-derived cells, which, in turn, can refer to embryonic stem cells (ESCs) or induced pluripotent stem cells (iPSCs). In some instances, the cardiovascular progenitor cells are human cells.

This disclosure describes methods of producing endocardial cells from cardiovascular progenitor cells (or, e.g., from pluripotent stem cells if the presence of BMP4, bFGF and ActivinA agonists are included). Endocardial cells can be identified by the expression of NKX2-5 and CD31, and can be produced by contacting pluripotent stem cell-derived cardiovascular progenitor cells with an FGF agonist and a BMP agonist under appropriate culture conditions. For example, endocardial cells can be generated by first differentiating pluripotent stem cells into cardiovascular mesoderm using a bFGF agonist, a BMP4 agonist, and an ActivinA agonist. Cardiovascular mesoderm then can be cultured in the presence of a bFGF agonist to generate cardiovascular progenitor cells, which then can be cultured in the presence of a bFGF agonist and a BMP10 agonist to specify an NKX2-5+ CD31+ endocardial population. In addition to NKX2-5+ CD31+, the population of endocardial cells described herein also expresses GATA4, GATA5, NFATC1, NPR3, NRG1, and ENG(CD105).

Representative FGF agonists are FGF2 (also known as basic FGF or bFGF), FGF1, FGF4, FGF8, FGF9, FGF10, FGF16 and FGF20, and representative BMP agonists include a BMP10, BMP9, BMP4, or BMP2 polypeptide. In some instances, the culture conditions appropriate to produce endocardial cells includes the absence of, or low levels (0-30 ng/mL) of, a VEGFA agonist. In some instances, the culture conditions appropriate to produce endocardial cells includes the absence of a Wnt inhibitor.

As the endocardial cells mature, NKX2-5 can be downregulated, giving rise to a NKX2-5− CD31+ population. It would be understood that NKX2-5− CD31+ cells derived from the NKX2-5+ CD31+ population are endocardial cells, but it would be appreciated that NKX2-5− CD31+ cells not derived from the NKX2-5+ CD31+ population (e.g., cultured in the presence of a FGF agonist and in the absence of a BMP10 agonist; or cultured in the presence of a FGF agonist and a VEGFA agonist) are endothelial cells and not endocardial cells.

Endocardial cells, or a population of endocardial cells, made by the methods described herein can be maintained by culturing them in serum free-media (e.g., StemPro) supplemented with a VEGFA agonist and a BMP10 agonist. The endocardial cells described herein also can be used to produce neuregulin (i.e., from the NRG1 gene), which is a cytokine.

### Valvular Interstitial-Like Cells (VICs)

Methods of making valvular interstitial-like cells (VICs) also are described herein. As described herein, the population of endocardial cells described herein includes valve progenitor cells having a PDGFRb+ phenotype. Such valve progenitor cells can be cultured in the presence of an FGF agonist, a BMP agonist, and a TGFbeta agonist. The resulting VICs have a phenotype of SOX9+, MSX2+, VIM+, VCAN+, COL1A1+, COL3A1+, POSTN+, CDH11+, NR4A2+, PRRX2+, TIMP3+, RGS5+ and ITGA2+.

When generating VICs, a representative FGF agonist is bFGF, representative BMP agonists include, without limitation, BMP2 or BMP4, and representative TGFbeta agonists include, for example, TGFbeta1, TGFbeta2 or TGFbeta3.

VICs produced as described herein can be used to model valvular heart disease and can be seeded on a biomimetic construct to generate a living tissue engineered heart valve for therapeutic applications.

### Cells Having a Trabecular Cardiomyocyte Fate

Methods of producing cells having a trabecular cardiomyocyte fate also are described herein. As described herein, immature ventricular cardiomyocytes can be cultured with the population of endocardial cells described herein, or with neuregulin produced from such a population of endocardial cells, such that the immature ventricular cardiomyocytes adopt a trabecular fate. The immature ventricular cardiomyocytes that can be used in this method typically are CTNT+ and can be derived, for example, from ventricular mesoderm. A trabecular phenotype, or cells having a trabecular fate, generally exhibit an upregulation of BMP10, NPPA, NPPB, IRX3, GJA5 and MYL2, and also can exhibit a downregulation of HEY2.

To produce cells having a trabecular fate, the combination of the population of endocardial cells, or neuregulin produced from such cells, and the immature ventricular cardiomyocytes can be cultured in the presence of a VEGFA agonist for about 4 to 5 days, after which trabecular marker expression in the cardiomyocytes can be maintained using neuregulin. Representative VEGFA agonists include, without limitation, VEGFA.

Cells having a trabecular fate as described herein can be maintained by culturing them in media containing neuregulin or with endocardial cells producing neuregulin in the presence of VEGFA to maintain endocardial cell identity, and can be used to generate cardiac Purkinje fibers in vitro. Purkinje fibers are the end point to which electrical signal arrives from the heart pacemakers (sinoatrial node and AV-node) and stimulates ventricular contraction. Purkinje fibers have been implicated in both the maintenance and the initiation of arrhythmias in the heart ventricles, which makes them an important cell type for modelling disease and for drug screening. In addition, the presence of cells exhibiting a trabecular fate can be used as an indicator of endocardial function.

### Biological Valves and Replenishing or Repairing Coronary Vasculature in Myocardium

A population of endocardial cells as described herein and/or the VICs described herein can be used in the construction of a biological valve, and such biological valves (e.g., made using the endocardial cells and/or the VICs described herein, or including cells differentiated from the endocardial cells and/or the VICs described herein) are provided. In addition, methods of replenishing or repairing coronary vasculature in myocardium (e.g., damaged myocardium) are provided, which include delivering the endocardial cells described herein to myocardium.

The endocardial cells described herein can be used to replenish coronary vasculature in myocardium. For example, the endocardial cells described herein and/or cells produced from such endocardial cells (e.g., coronary endothelial cells) can be delivered to heart tissue (e.g., via injection). Upon engraftment, the cells then can revascularize the myocardium. Such therapeutic applications of the cells described herein can be beneficial when the heart tissue is damaged.

Methods are being developed to transplant cardiomyocytes in order to remuscularize damaged myocardium. Current strategies are focused on transplanting cardiomyocytes alone, however, this approach is inefficient, in part, because many of the transplanted cardiomyocytes do not survive. However, the transplantation of coronary endothelial cells generated from the endocardial cells described herein together with cardiomyocytes can improve, in some cases significantly, the efficiency of engraftment (e.g., using fewer cells, with larger grafts). It would be appreciated that each of types of cells (i.e., the endocardial cells and the cardiomyocytes) can be delivered (e.g., transplanted) separately or together, and can be delivered using, for example, injection (e.g., co-injection).

### Screening Methods

The endocardial cells described herein, the VICs described herein, or the cells having a trabecular fate described herein can be used to screen test compounds to identify those that exhibit toxicity. The endocardial cells described herein, the VICs described herein, or the cells having a trabecular fate described herein can be contacted with test compounds from classes such as proteins, small molecules, or nucleic acids, and the viability of the cells determined. Those compounds that reduce the viability of the cells can be deemed to have some toxicity against such cells.

Methods of determining the viability of cells are known in the art and include, for example, assays for apoptosis (e.g., Annexin V, TUNEL, or caspase) or for cell proliferation (e.g., methyl violet, neutral red uptake, trypan blue, BrdU).

The endocardial cells described herein can be used in vitro to study valve diseases. For example, endocardial cells can be produced using the methods described herein from patients having a valve disease (e.g., hypoplastic left heart syndrome (HLHS)) in order to study the etiology of the disease and/or the effects of one or more test compounds.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Generation and Maintenance of Human ESC/iPSC Lines

The generation of the HES3-NKX2-5eGFP/w (karyotype: 46, XX) and HES2:RFP hESC (karyotype: 46, XX) reporter cell lines was described previously (Irion et al., 2007, Nat. Biotechnol., 25(12):1477-82; Elliott et al., 2011, Nat. Methods, 8(12):1037-40). The HES2:GFP hESC line was generated from the HES2:RFP hESC line by exchanging the tdRFP cassette with a EGFP expressing cassette. The hPSC lines were maintained on irradiated mouse embryonic fibroblasts in hPSC culture media consisting of DMEM/F12 (Cellgro), penicillin/streptomycin (1%, ThermoFisher), L-glutamine (2 mM, ThermoFisher), non-essential amino acids (1×, ThermoFisher), beta-mercaptoethanol (55 μM, ThermoFisher) and KnockOut™ serum replacement (20%, ThermoFisher) as described previously (Kennedy et al., 2007, Blood, 109(7):2679-87).

### Example 2—Directed Differentiation of Human PSC Lines Into Cardiomyocytes

For cardiac differentiation, a previously described protocol was used for generating ventricular cardiomyocytes from mesoderm induced with 10 ng/ml BMP4 and 6 ng/ml activin A (Lee et al., 2017, Cell Stem Cell., 21(2):179-94 e174). To initiate differentiation, hPSCs at 80%-90% confluence were dissociated into single cells (TrypLE, ThermoFisher) and re-aggregated to form EBs at a cell density of 5×10e5 cells/ml in StemPro-34 media (ThermoFisher) supplemented with penicillin/streptomycin (1%, ThermoFisher), L-glutamine (2 mM, ThermoFisher), ascorbic acid (50 μg/ml, Sigma), monothioglycerol (50 μg/ml, Sigma), transferrin (150 μg/ml, ROCHE), ROCK inhibitor Y-27632 (10 μM, TOCRIS) and rhBMP4 (1 ng/ml, R&D). For EB generation, the cultures were rotated for 18 h on an orbital shaker (MaxQ 2000 shaker, Thermofisher) in 6 cm petri dishes (VWR) at 60 RPM. Following the rotation step, the EBs were transferred to fresh 6 cm petri dishes (VWR) in mesoderm induction media consisting of StemPro-34 supplemented with penicillin/streptomycin (1%), L-glutamine (2 mM), ascorbic acid (50 μg/ml), monothioglycerol (50 μg/ml), transferrin (150 μg/ml), rhBMP4 (10 ng/ml, R&D), rhActivinA (6 ng/ml, R&D) and rhbFGF (5 ng/ml, R&D). At day 3, the EBs were harvested, washed with IMDM and transferred to cardiac specification media consisting of StemPro-34 supplemented with penicillin/streptomycin (1%), L-glutamine (2 mM), ascorbic acid (50 μg/ml), monothioglycerol (50 μg/ml), transferrin (150 μg/ml), the Wnt inhibitor IWP2 (2 μM, TOCRIS) and rhVEGFA (5 ng/mL, R&D). At day 5, the EBs were harvested, washed with IMDM and cultured in StemPro-34 supplemented with penicillin/streptomycin (1%), L-glutamine (2 mM), ascorbic acid (50 μg/ml), monothioglycerol (50 μg/ml), transferrin (150 μg/ml) and rhVEGFA (5 ng/ml) for another 4 days. EBs cultures for periods longer than 9 days were maintained in StemPro-34 supplemented with penicillin/streptomycin (1%), L-glutamine (2 mM), ascorbic acid (50 μg/ml) and monothioglycerol (50 μg/ml). The media was changed every 2 days. At days 9, 16 or 23 HES2:RFP− or HES2:GFP-derived cardiomyocytes were isolated based on the expression of SIRPA (Dubois et al., 2011, Nat. Biotechnol., 29(11):1011-8). To generate cardiomyocyte aggregates, sorted cells were plated at a density 5×10e4 cells per well in 96-well flat bottom ultra-low attachment microplates (Corning) for 8 days in StemPro-34 supplemented with penicillin/streptomycin (1%), L-glutamine (2 mM), ascorbic acid (50 μg/ml), monothioglycerol (50 μg/ml) with or without NRG1 (50 ng/ml). The media was changed every 2 days. The cultures were incubated in a low oxygen environment (5% CO2, 5%O2, 90% N2) for first 9 days and a normoxic environment (5% CO2) after day 9.

### Example 3—Generation of Endocardial/Endothelial Cells

To generate endocardial/endothelial cells, hPSCs were differentiated to cardiogenic mesoderm using the protocol described above. At day 3, the EBs were dissociated with TrypLE for 3 min at 37° C. and the cells plated in 96-well flat bottom microplates (Falcon) coated with Matrigel (25% v/v, Corning) at a density of 10e5 cells per well. The monolayers were cultured in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma), transferrin (150 μg/ml, ROCHE) and rhbFGF (50 ng/ml, R&D). For endocardial induction, rhBMP10 (10 ng/ml) was added to the media from day 5 to day 9. For generation of control endothelial cells, VEGFA (100 ng/mL, R&D) was added to the differentiation media from day 3 to day 9. The cultures were incubated in a low oxygen environment (5% CO2, 5%O2, 90% N2) and the media was changed every 2 days. At day 9, HES3-NKX2-5eGFP/w-derived endocardial/endothelial cells were analyzed and isolated based on the expression of NKX2-5:GFP and CD31. The endocardial cells generated from the non-transgenic hPSC lines were analyzed and isolated as CD31+ populations. For dissociation, the monolayers were incubated in Collagenase type 2 (1 mg/ml, Worthington) in HANKs buffer at 37° C. for one hour.

### Example 4—Co-Culture of Endocardial/Endothelial With Cardiomyocytes

For culture in the absence of cardiomyocytes, the endocardial or control endothelial cells (HES3-NKX2-5eGFP/w line) isolated at day 9 of differentiation were plated in a 96-well flat bottom microplate (Falcon) coated with Matrigel (25% v/v, Corning) at a density 2.5×10e4 cells per well in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma), rhVEGFA (100 ng/ml, R&D) with or without rhBMP10 (10 ng/ml) or rhBMP4 (10 ng/ml). For co-culture of the two cell types, 5×10e4 hPSC-derived SIRPA+ cardiomyocytes (day 9) were mixed together with either 2.5×10e4 HES3-NKX2-5eGFP/w- or 5×10e4 HES2:RFP-derived endocardial or endothelial cells and cultured for 8 days in 96-well flat bottom microplates (Falcon) coated with Matrigel (25% v/v, Corning) in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma) and rhVEGFA (100 ng/ml, R&D). For inhibition of the NRG-ERBB pathway lapatinib (10 μM, LC labs) was added to the media. Control cardiomyocytes were cultured as aggregates as described above in the same media with or without NRG1 (50 ng/ml). The cultures were incubated in a normoxic environment (5% CO2) and the media was changed every 2 days.

### Example 5—Generation of Mesenchymal/Valvular Interstitial-Like Cells From Endocardial/Endothelial Cells

Day 9 endocardial/endothelial bulk cultures (HES3-NKX2-5eGFP/w line) were dissociated with Collagenase type 2 as described above. Single cell suspensions were enriched for CD31+ CD34+ cells by magnetic activated cell sorting (MACS, Miltenyi, 130-146-702) to a purity of over 95%. Cells were incubated with anti-CD34 microbeads for 30 minutes at 4° C. in base media supplemented with DNAse (1 U/ml, Millipore)(10 μl microbeads/5×10e6 cells in 100 μl of media) and purified by MS or LS columns. After MACS cells from endocardium were further sub-fractionated by FACS into CD31+ CD34+ PDGFRb+ and CD31+ CD34+ PDGFRb− populations. After the sort, total CD31+ CD34+, CD31+ CD34+ PDGFRb+ and CD31+ CD34+ PDGFRb− endocardial cells as well as total CD31+ CD34+ endothelial cells were plated in a 12-well flat bottom microplate (Falcon) coated with Matrigel (25% v/v, Corning) at a density 8×10e5 cells per well and cultured for 8 days in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma), rhbFGF (10 ng/ml, R&D), rhBMP2 (100 ng/ml), TGFbeta2 (0.3 ng/ml, R&D).

For enrichment of CD31− PDGFRB+ VIC-like cells, MACS-sorted CD31+ CD34+ endocardial-like cells were plated in a 12-well flat bottom microplate (Falcon) coated with Matrigel (25% v/v, Corning) at a density 2×10e5 cells per well and cultured for 12 days in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma) in the presence of rhbFGF (10 ng/ml, R&D), rhBMP2/4 (100 ng/ml), CHIR99021 (1 μM), SB-431542 (5.4 μM) for 4 days, followed by rhbFGF (10 ng/ml, R&D), rhBMP2/4 (100 ng/ml), TGFbeta2 (0.3 ng/ml, R&D) for 8 days.

### Example 6—Generation of Coronary Endothelial-Like Cells From Endocardial Cells

Day 9 endocardial/endothelial bulk cultures (HES3-NKX2-5eGFP/w line) were dissociated with Collagenase type 2 as described above. Single cell suspensions were enriched for CD31+ CD34+ cells by magnetic activated cell sorting (MACS, Miltenyi, 130-146-702) to a purity of over 95% as for VIC protocol. Cells were plated in a 12-well flat bottom microplate (Falcon) coated with Matrigel (25% v/v, Corning) at a density 4×10e5 cells per well and cultured for 8 days in StemPro-34 supplemented with penicillin/streptomycin (1%, ThermoFisher), L-glutamine (2 mM, ThermoFisher), ascorbic acid (50 μg/ml, Sigma), monothioglycerol (50 μg/ml, Sigma) in the presence of rhVEGFA (50 ng/ml, R&D) for 4 days, followed by rhVEGFB (50 ng/ml, R&D) and PPARa agonist GW7647 (1 μM) for additional 4 days.

### Example 7—Flow Cytometry and Cell Sorting

Day 3 EBs were dissociated with TrypLE for 3 min at 37° C. Day 9-23 EBs or mixed populations of cardiomyocytes and endocardial/endothelial cells were dissociated by incubation in Collagenase type 2 (1 mg/ml, Worthington) in HANKS buffer at 37° C. for one hour followed by TrypLE treatment (5 min at 37° C.). The following antibodies were used for staining: anti-SIRPA-PeCy7 (Biolegend, 1:1000), anti-CD31-PE (BD Pharmingen, 1:100), anti-CD31-FITC (BD Pharmingen, 1:100), anti-CD31-AF647 (BD Pharmingen, 1:100), anti-CD90-APC (BD Pharmingen, 1:1000), anti-PDGFRB-BV421 (BD Pharmingen, 1:100), anti-CD105-APC (eBioscience, 1:200), anti-CD56-APC (BD Pharmingen, 1:100), anti-PDGFRA-PE (BD Pharmingen, 10:100), anti-CD36-APC (Biolegend, 1:100), anti-LDLR-BV421 (Biolegend, 1:100), anti-cardiac isoform of CTNT (ThermoFisher Scientific, 1:2000), or anti-myosin light chain 2 (Abcam, 1:1000), anti-ANP (Abcam, 1:500). For unconjugated primary antibodies, the following secondary antibodies were used for detection: goat anti-mouse IgG-APC (BD Pharmigen, 1:250), or donkey anti-rabbit IgG-AF488 (ThermoFisher Scientific, 1:1000). Detailed antibody information is described in the Key Resources Table. For cell-surface marker analyses, cells were stained for 30 min at 4° C. in FACS buffer consisting of PBS with 5% fetal calf serum (FCS) (Wisent) and 0.02% sodium azide. For intracellular staining, cells were fixed for 15 min at 4° C. with 4% PFA in PBS followed by permeabilization using 90% methanol for 20 min at 4° C. Cells were washed with PBS containing 0.5% BSA (Sigma) and stained with unconjugated primary antibodies in FACS buffer overnight at 4° C. Stained cells were washed with PBS with 0.5% BSA and stained with secondary antibodies in FACS buffer for 1 h at 4° C. Stained cells were analyzed using an LSR II Flow cytometer (BD). For cell sorting, cells were stained, washed and kept in IMDM with 0.2% KnockOut™ serum replacement and sorted using either Influx (BD), FACSAriaII (BD), MoFlo-XDP (BD) of FACSAria Fusion (BD) cells sorter at the Sickids/UHN flow cytometry facility. Data were analyzed using FlowJo software (Tree Star).

### Example 8—Immunohistochemistry

Day 3 EBs generated from HES3-NKX2-5eGFP/w line under 10B/6A mesoderm induction conditions were dissociated as described above and the cells plated onto 12 mm cover glasses (VWR) pre-coated with Matrigel (25% v/v, BD) in 24-well plates (Falcon) at a density 2×10e5 cells per well. The cells were cultured for 6 days as monolayers under endocardial, control endothelial or cardiomyocyte differentiation conditions. Following culture, the cells were fixed with 4% PFA in PBS for 10 min at room temperature and permeabilized with PBS containing 0.2% TritonX for 20 min at RT. The fixed cells were blocked with PBS containing 10% FCS and 2% BSA. The following antibodies were used for staining: rabbit anti-NKX2-5 (Cell Signaling, 1:100), rabbit anti-GATA4 (Abcam, 1:100), rabbit anti-NFAT2 (Abcam, 1:100), mouse anti human CD31 (Dako, 1:100), mouse anti-cardiac isoform of CTNT (ThermoFisher Scientific, 1:100), goat anti-GFP (Rockland, 1:500). For detecting unconjugated primary antibodies, the following secondary antibodies were used: donkey anti-mouse IgG-A647 (ThermoFisher, 1:1000), donkey anti-rabbit IgG-A555 (ThermoFisher, 1:1000) and donkey anti-goat IgG-A488 (ThermoFisher, 1:1000). Detailed antibody information is described in the Key Resources Table. The cells were stained with primary antibodies in staining buffer consisting of PBS with 0.05% TritonX and 2% BSA overnight at 4° C. The stained cells were washed with PBS containing 0.1% BSA 3× for 10 min each wash at room temperature. The cells were then stained with secondary antibodies in staining buffer for 1 h at room temperature followed by a wash step as described above. The cell nuclei were stained with DAPI (Biotium, 0.3 μg/ml) in wash buffer for 5 min at room temperature. Following staining, the samples were mounted using ProLong Diamond Antifade Mountant (ThermoFisher). The stained cells were analyzed using an Olympus FluoView 1000 Laser Scanning Confocal Microscope. FV10-ASW software was used for image acquisition.

### Example 9—Quantitative Real-Time PCR

Total RNA from hPSC-derived populations was isolated using RNAqueous-micro Kit including RNase-free DNase treatment (Ambion). Between 100 ng and 1 mg of isolated RNA was reverse transcribed into cDNA using oligo (dT) primers and random hexamers and Superscript III Reverse Transcriptase (ThermoFisher). RT-qPCR was performed on an EP Real-Plex MasterCycler (Eppendorf) using QuantiFast SYBR Green PCR kit (QIAGEN). All experiments were prepared in duplicates and included a 10-fold dilution series of sonicated human genomic DNA standards ranging from 25 ng/ml to 2.5 pg/ml for evaluating the efficiency of the PCR reaction and the copy number of each gene relative to the house keeping gene TBP. Heat maps of gene expression data were generated using the MultiExperiment Viewer (MeV) open source software. Primer sequences are listed in the Table below.

### Example 10—Quantification and Statistical Analysis

All data are represented as mean±standard error of mean (SEM). Sample sizes (n) represent biological replicates of differentiation experiments. No statistical method was used to predetermine the samples size. Due to the nature of the experiments, randomization was not performed and the investigators were not blinded. Statistical significance was determined by using Student's t test (paired, two-tailed) and one-way or two-way ANOVA analysis with Bonferroni post-hoc test in GraphPad Prism 7 software. Results were considered to be significant at p<0.05 (*), p<0.005 (**), p<0.0005 (***). All statistical parameters are reported in the respective figures and figure legends.

### Example 11—FGF and BMP Induce NKX2-5 Expression in Cardiogenic Mesoderm

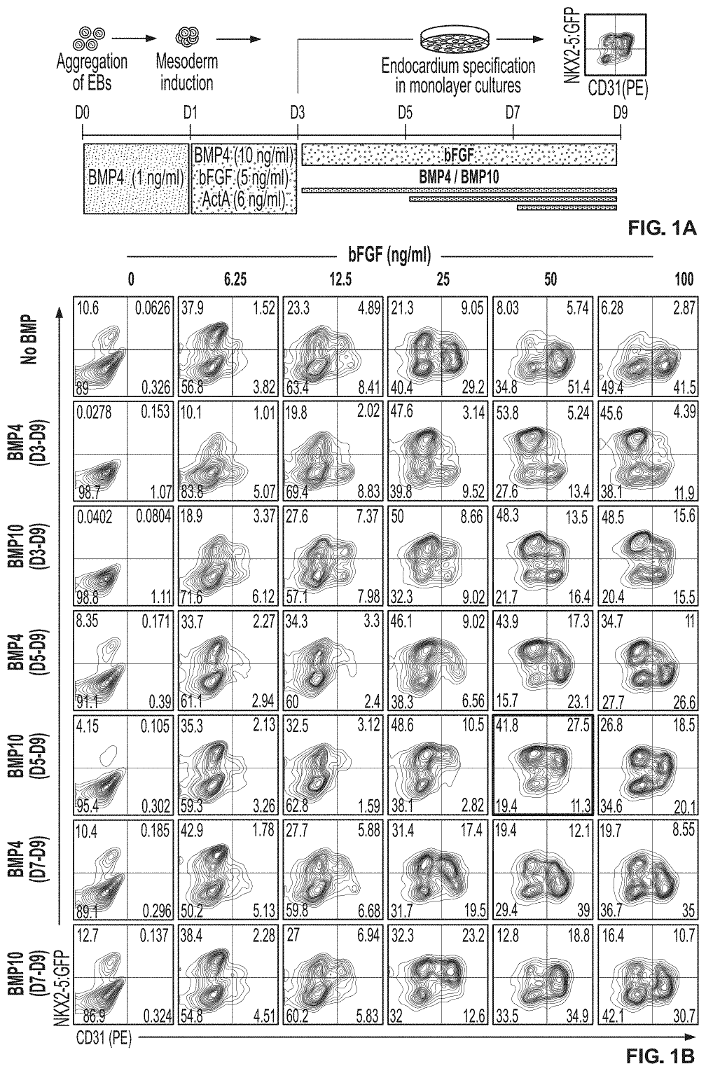

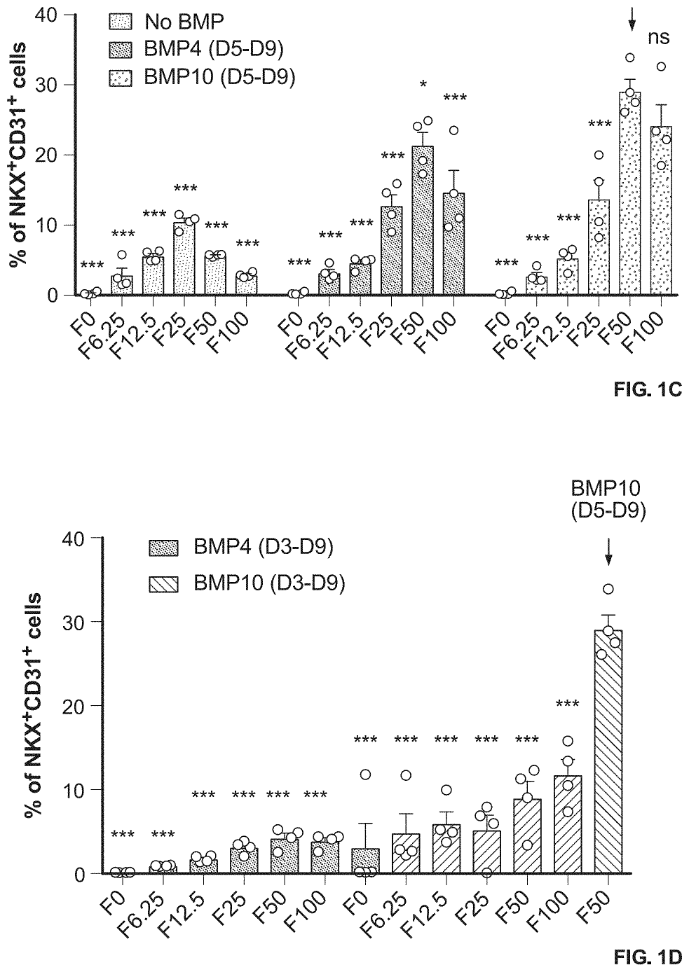

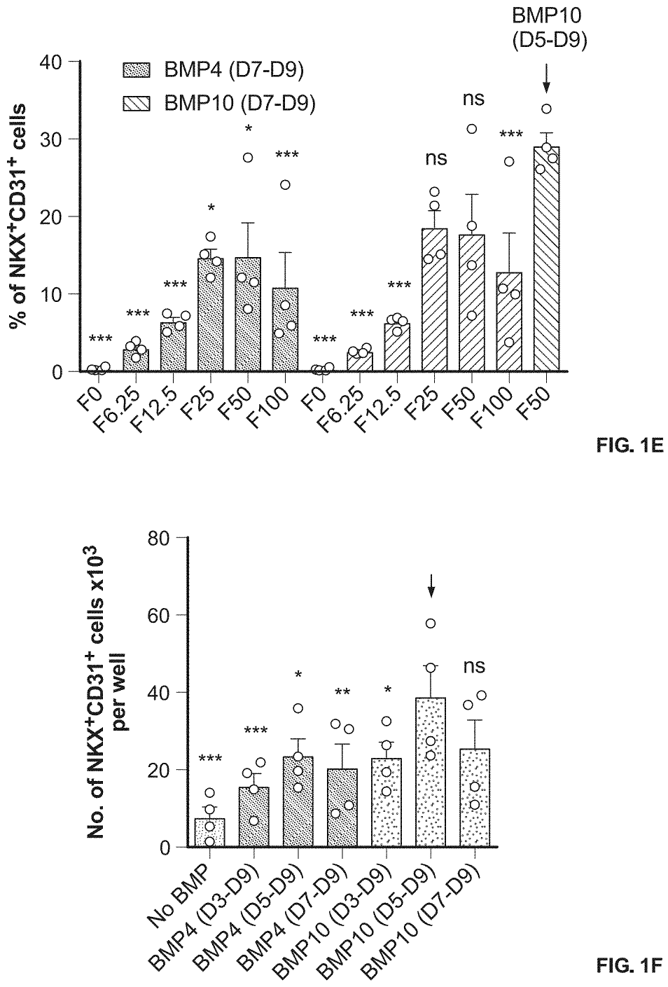

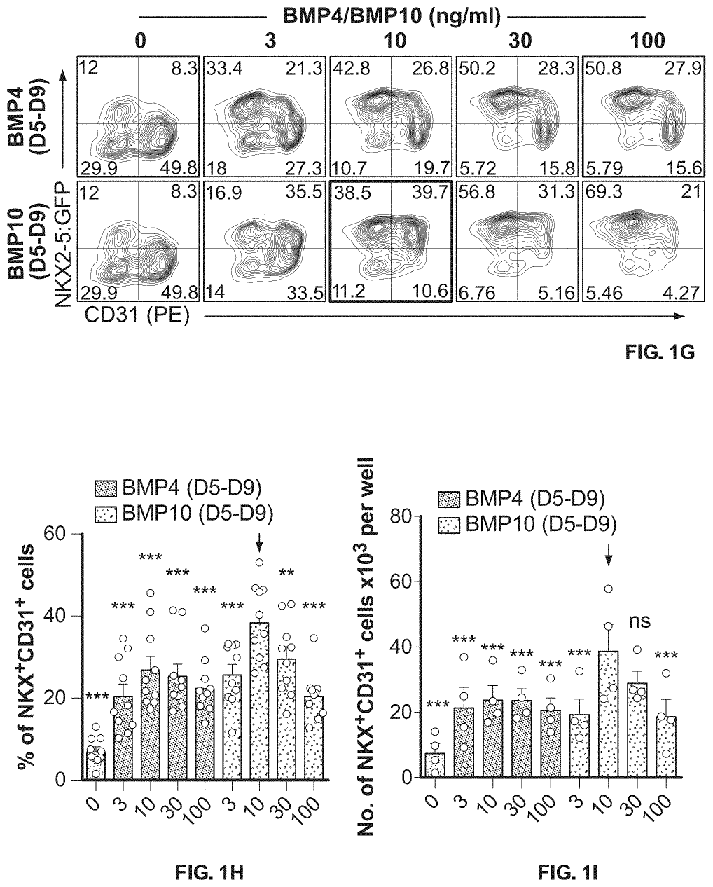

Initial experiments were set up to identify the signaling pathways that would promote the generation of a NKX2-5+ CD31+ cell that we propose represents the earliest stage of human endocardial development. The transgenic HES3-NKX2-5eGFP/w reporter line (Elliott et al., 2011, Nat. Methods, 8(12):1037-40) was used to allow for the identification of NKX2-5+ cells by flow cytometry. As previous studies have shown that both FGF and BMP signaling are involved in the specification of NKX2-5+ cardiac progenitors in vivo, the role of these pathways in the generation of the target NKX2-5+ CD31+ population in the hPSC differentiation cultures was evaluated. For these studies, either BMP4 or BMP10 was used, as both are expressed in the early cardiomyocytes that develop adjacent to the endocardial cells. These pathways were manipulated in cardiogenic mesoderm induced with the concentrations of activin and BMP4 (6 ng/ml activin, 10 ng/ml BMP4) used in previous studies to generate ventricular cardiomyocytes (FIG. 1A). To optimize exposure of the mesoderm to these factors, the day 3 EBs were dissociated and the cells plated as a monolayer on matrigel treated plastic.

In the first set of experiments, the effects of varying the concentration of bFGF between days 3 and 9 of differentiation were evaluated in the presence of either BMP4 or BMP10 that were added in the following time intervals: days 3-9, 5-9 or 7-9. These times were chosen to match the emergence of cardiomyocytes (between days 6 and 9) in the cultures, in an effort to recapitulate the coordinated development of the endocardial and cardiomyocyte lineages in the early embryo. For the initial analyses, a single concentration of BMP4 or BMP10 was used (10 ng/ml). At day 9, the populations were harvested and analyzed for the presence of NKX2-5+ and CD31+ cells. None of the populations induced with BMP4 or BMP10 alone in the absence of bFGF contained CD31+ cells, indicating that exogenous FGF signaling enhances endothelial development under these conditions (FIG. 1B). A NKX2-5+ CD31− population was detected in some of the groups. bFGF alone induced three different NKX2-5 and CD31 populations, although the size varied with the concentration of the factor added. Low concentrations of FGF favored the generation of NKX2-5+ CD31− cells, whereas higher concentrations promoted the development of NKX2-5− CD31+ cells. None of the concentrations of bFGF induced large NKX2-5+ CD31+ populations. When paired with exogenous FGF signaling, addition of either BMP4 or BMP10 significantly increased the proportion of NKX2-5+ CD31+ cells that were generated, with the highest frequency detected in the groups induced with concentrations of bFGF of 25 ng or greater. The addition of BMP from days 5-9 induced a higher proportion of NKX2-5+ CD31+ than the extended addition of agonist from day 3-9 (FIGS. 1C, 1D). Within the day 5-9 time frame, the highest frequency of these cells was induced by the combination of BMP10 and 50 or 100 ng/ml of FGF-beta (FIG. 1C). Addition of BMP from days 7-9 promoted the development of comparable frequencies of NKX2-5+ CD31+ cells as observed in the populations treated from days 5-9 (FIG. 1E). However, this delayed addition led to the development of a larger NKX2-5− CD31+ subpopulation in the BMP10/FGF50− or BMP10/FGF100-induced populations. Based on these observations and on the number of NKX2-5+ CD31+ cells generated (FIG. 1F), the combination of 50 ng/ml of bFGF and BMP10 added from days 5-9 was chosen for the following experiments.

The effects of different concentrations of BMP on the induction of the NKX2-5+ CD31+ population at a defined bFGF dose was determined. As shown in FIG. 1G-1I, the highest frequency and number of NKX2-5+ CD31+ cells was induced by the addition of 10 ng/ml of BMP10.

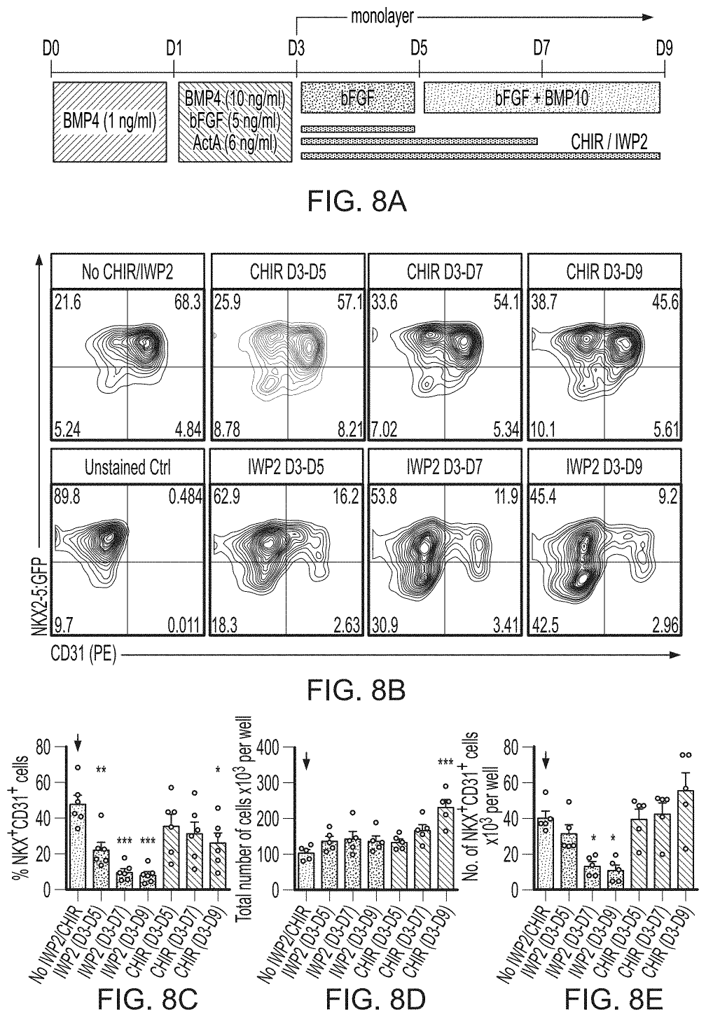

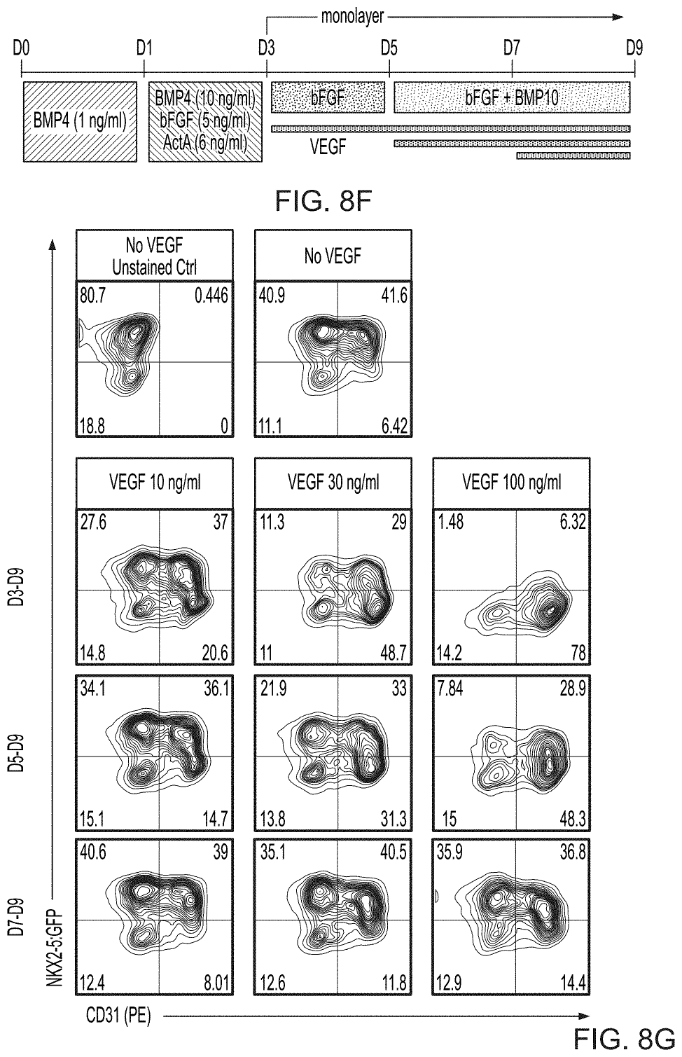

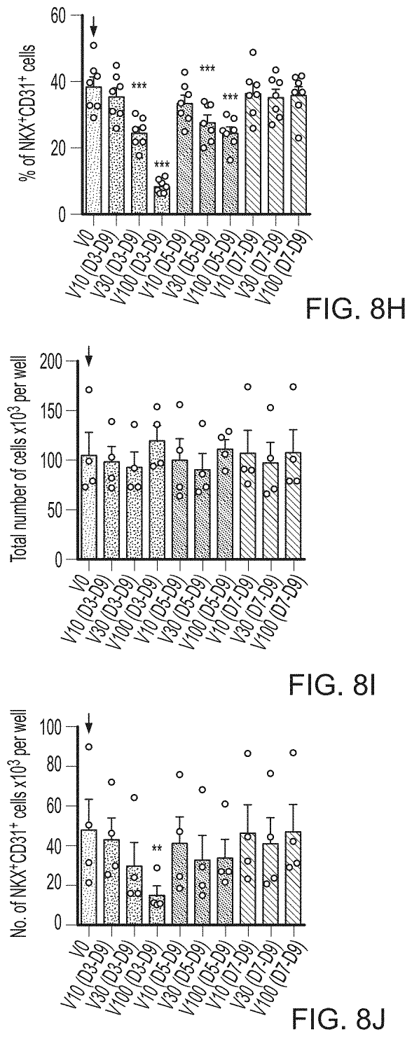

The protocol for cardiomyocytes specification from hPSC-derived cardiovascular mesoderm included a WNT inhibition step from day 3 to day 5. As the same mesoderm was used to generate the NKX2-5+ CD31+ cells in the above studies, we were next interested in determining if WNT signaling plays a role in development of these cells. To address this, the WNT pathway was either activated through the addition of the small molecule GSK-3 inhibitor, CHIR99021, or inhibited by the addition of IWP2 for 2, 4 or 6 days, beginning at day 3 of differentiation (FIGS. 8A-8E). Inhibition of WNT during each of these different time intervals resulted in a significant reduction in the frequency of NKX2-5+ CD31+ generated. The total number of these cells was reduced by the addition of the inhibitor for 4 or 6 days. The addition of CHIR did not impact the total number of NKX2-5+ CD31+ produced in the cultures, but addition for 6 days led to a decrease in their frequency. Together, these findings indicate that WNT signaling is required for the formation of NKX2-5+ CD31+ cells and that the amount produced endogenously by the differentiating cells is sufficient for their development. Next, we investigated the role of exogenous VEGFA/KDR signaling on the generation of the NKX2-5+ CD31+ population as the day 3 mesoderm and derivative cells express low levels of KDR. Addition of different concentrations of VEGFA during the time intervals outlined in FIG. 8F did not increase the number of NKX2-5+ CD31+ cells generated (FIG. 8G, 8J). Rather, when added for the 3-9 day window, the highest concentration of agonist led to a significant decrease in the total number of NKX2-5+ CD31+ cells produced and a corresponding increase in the number of NKX2-5− CD31+ cells (FIGS. 8G-8J). Given these observations, VEGFA was not included in the protocol for the generation of NKX2-5+ CD31+ cells for the studies described below.

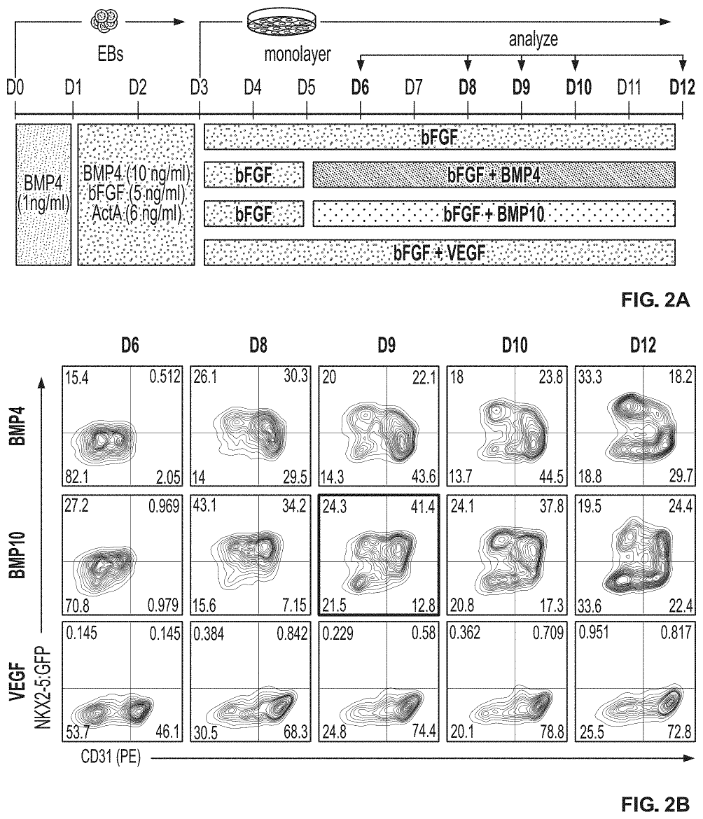

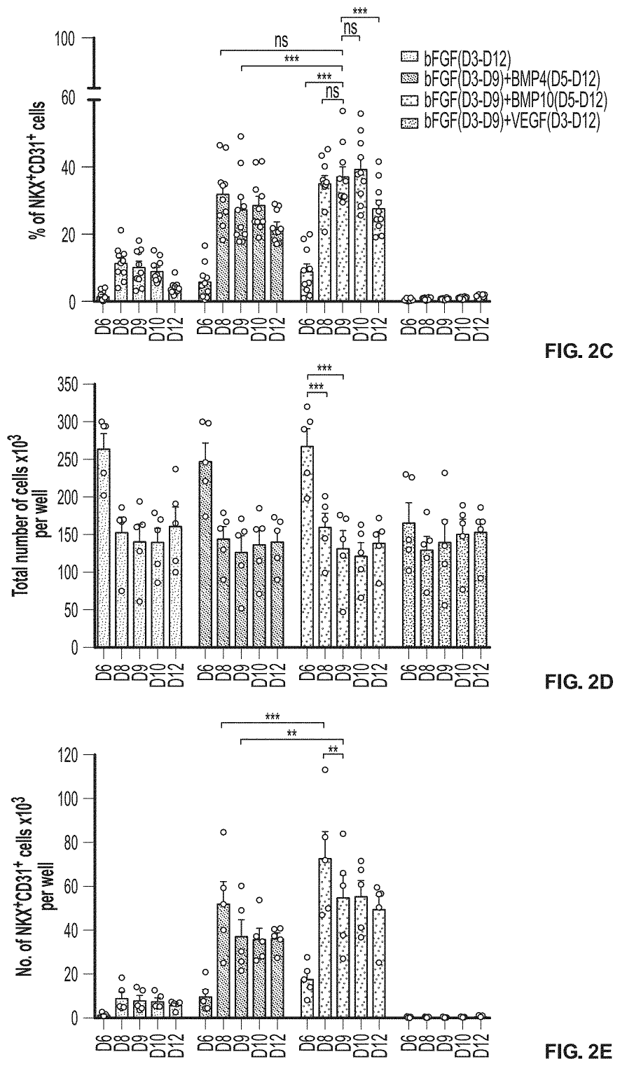

The kinetics of NKX2-5+ CD31+ development was analyzed by comparing BMP4 to BMP10 induction using the concentrations of factors and timing of their addition determined in the above experiments (FIG. 2). In the presence of both BMP agonists, NKX2-5+ CD31+ cells emerged at day 8 and the populations persisted to day 12. Although the frequency of NKX2-5+ CD31+ cells that developed did not differ between the two groups at day 8, the total number of these cells generated at days 8 and 9 in the BMP10-induced populations was significantly higher than in the BMP4-treated populations (FIG. 2E).

Exploiting the above observation that high concentrations of VEGFA promotes the development of a NKX2-5− CD31+ population, the cells were treated with VEGFA and bFGF without BMP, in an effort to generate NKX2-5− CD31+ non-endocardial endothelial cells. As shown in FIG. 2B, the addition of VEGFA/bFGF from day 3-9 induced a NKX2-5− CD31+ population that could be detected as early as day 6 and persisted to day 12 of differentiation. As expression of NKX2-5 was not upregulated at any stage under these conditions, it was reasoned that these cells represent non-endocardial endothelial cells and they were used as ‘control’ cells in the following studies.

Given the findings from these kinetic analyses and the fact that the protocol was optimized in the previous experiments for a day 9 population, the NKX2-5+ CD31+ cells from day 9 cultures were isolated for the following studies.

### Example 12—Characterization of NKX2-5+ CD31+ Cells

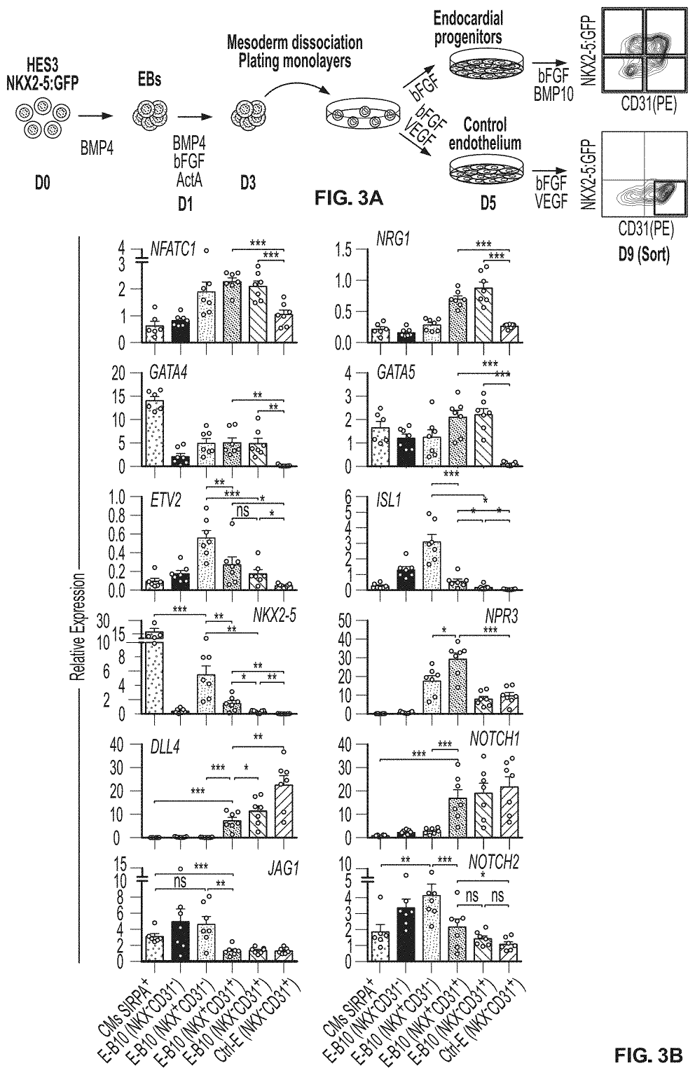

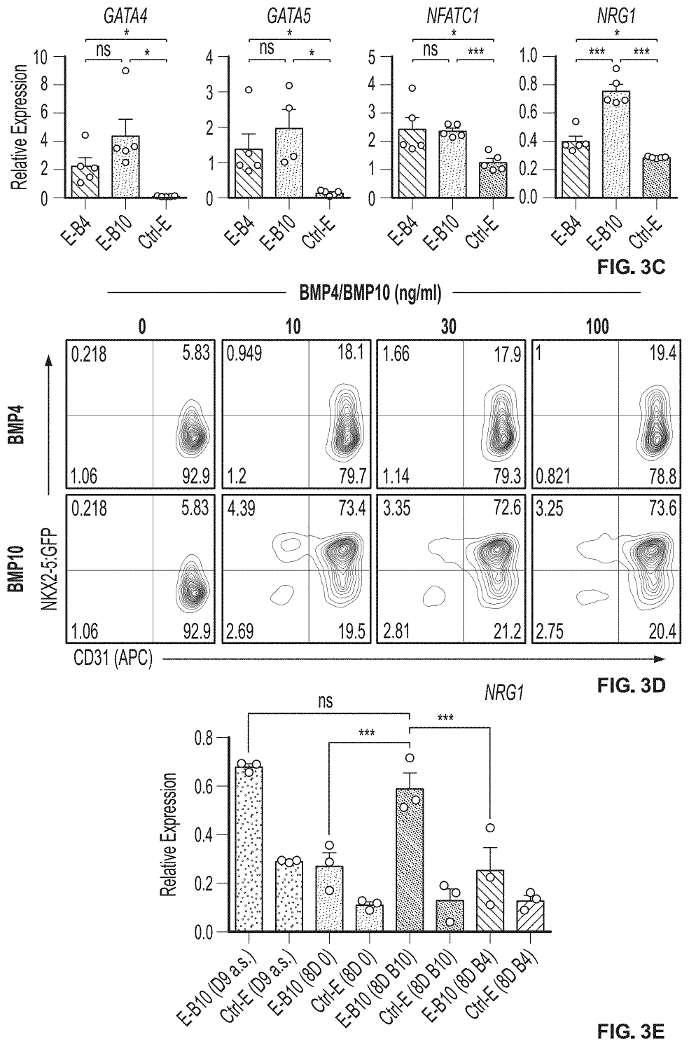

Endocardial cells can be distinguished from other endothelial cells by the levels of expression of specific transcription factors including GATA4, GATA5 and NFATC1 as well as by the expression of atrial natriuretic peptide receptor, NPR3, and neuregulin (NRG1), the ligand to the ERBB family receptors (de la Pompa et al, 1998, Nature, 392(6672):182-6; Ranger et al., 1998, Nature, 392(6672):186-90; Charron and Nemer, 1999, Semin. Cell Dev. Biol., 10(1):85-91; Nemer and Nemer, 2002, Development, 129(17):4045-55; Rivera-Feliciano et al., 2006, Development, 133(18):3607-18; Wu et al., 2013, Trends Cardiovasc. Med., 23(8):294-300; Zhang et al., 2016, Circ. Res., 118(12):1880-93). To further characterize the NKX2-5+ and CD31+ populations generated with the optimized protocol, the different populations were isolated by Fluorescence-Activated Cell Sorting (FACS) and analyzed for the expression of these genes (FIG. 3A). Analyses of NKX2-5, ISL1 and ETV2 that are expressed in the endocardial progenitors also was included. The expression patterns were compared to the control endothelial cells induced in the absence of exogenous BMP signaling. As shown in FIG. 3B, the NKX2-5+ CD31+ cells expressed significantly higher levels of all of the genes compared to control endothelial population. The levels of expression of NFATC1, NRG1, GATA4 and GATA5 were similar in the NKX2-5+ CD31+ and the NKX2-5-CD31+ populations whereas the levels of NPR3 were significantly higher in the NKX2-5+ CD31+ cells. These findings suggest that both populations contain endocardial-like cells. The differences in NPR3 expression may reflect differences in the stage of maturation. The NKX2-5+ CD31 cells expressed higher levels of the progenitor markers, NKX2-5, ETV2 and ISL1 than the NKX2-5+ CD31+ and NKX2-5− CD31+ cells suggesting that this population contains the endocardial progenitors. Given that DLL4-NOTCH1 signaling in endocardium is important for trabeculation of the heart tube (Grego-Bessa et al., 2007, Dev. Cell, 12(3):415-29; D'Amato et al., 2016, Nat. Cell Biol., 18(1):7-20), these population also were analyzed for the expression of NOTCH ligands and receptors. The NKX2-5+ CD31+, NKX2-5− CD31+ and control population expressed DLL4 and NOTCH1 at higher levels than the NKX2-5+ CD31 population, whereas the reverse patterns were observed for JAG1 and NOTCH2 (FIG. 3B). Taken together, the findings from these molecular analyses are consistent with the interpretation that the BMP10-induced NKX2-5+ CD31+ and NKX2-5− CD31+ populations represent the human endocardial lineage.

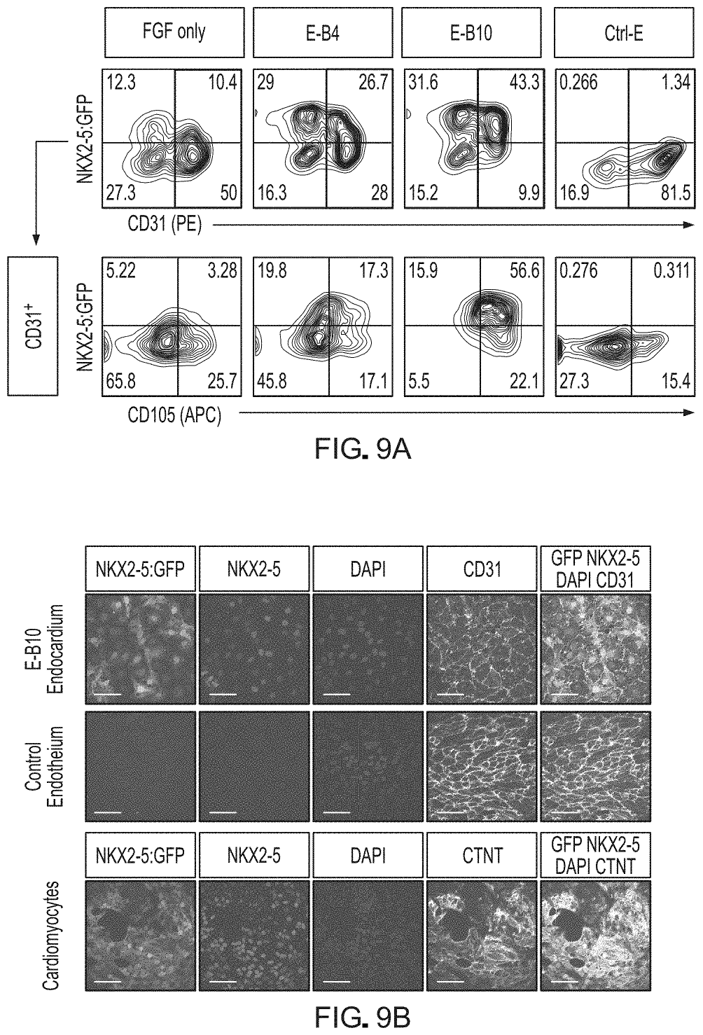

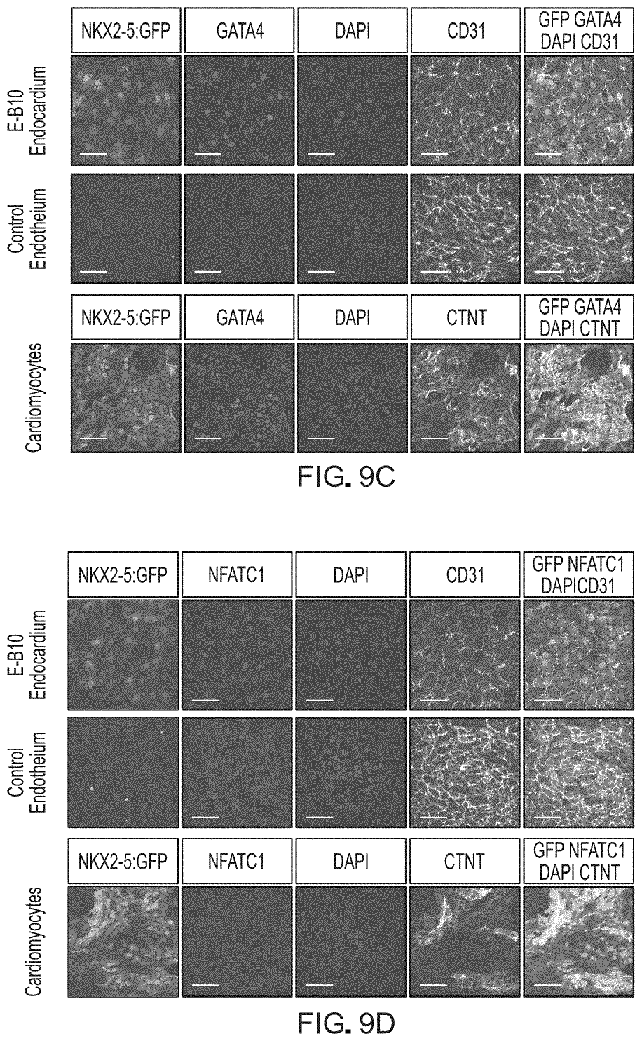

To determine if the method of induction impacted the molecular profile of the NKX2-5+ CD31+ population, the expression patterns in cells induced with either BMP4 or BMP10 was compared. Although the levels of expression of GATA4, GATA5 and NFATC1 were similar in both, the BMP10-induced population expressed higher levels of NRG1 than the cells induced with BMP4 (FIG. 3C). The two populations also were analyzed for the expression of CD105 (endoglin), which has been shown to be involved in valve formation. As shown in FIG. 9A, the BMP10-induced population contained a much larger fraction of CD105+ cells than the BMP4-induced or control endothelial populations.

Collectively, the findings from the above comparative studies demonstrate that BMP signaling plays a pivotal role in the generation of NKX2-5+ CD31+ cells that express markers indicative of endocardial cells. Additionally, they suggest that BMP10 is more effective than BMP4 in specifying this fate. To further investigate differences between these pathway agonists, BMP10-induced NKX2-5+ CD31+ cells were cultured in the presence of different concentrations of either BMP4 or BMP10 for 8 days. VEGFA was added to maintain the endothelial phenotype of the cells during this culture period. Analyses of the cultured populations revealed that BMP10 was more effective than BMP4 in maintaining the NKX2-5+ CD31+ cell phenotype at all concentrations of agonist tested (FIG. 3D). The cells cultured in BMP10 also sustained higher levels of NRG1 than those maintained in BMP4 (FIG. 3E). Together, these findings demonstrate that BMP10 signaling is essential for maintaining endocardial-like cells in cultures. Notably, the addition of BMP10 to the control endothelial population during this culture period did not lead to the upregulation of NRG1 expression, indicating that this agonist does not directly upregulate NRG1 in non-endocardial endothelial cells.

To further characterize the BMP10-induced NKX2-5+ CD31+ endocardial-like cells, the cells were analyzed for the presence of NKX2-5, GATA4 and NFATC1 proteins by immunostaining analyses. As shown in FIGS. 9B-9D, these cells as well as the hPSC-derived cardiomyocytes contained NKX2-5 and GATA4 protein, consistent with the RT-qPCR expression profiles. The control endothelial cells, by contrast, showed no expression. NFATC1 was detected in the nuclei of NKX2-5+ CD31+ endocardial-like cells as well as in the control endothelial cells, indicating that this marker alone does not distinguish these populations.

### Example 13—NRG1 Induces Markers of Trabecular Myocardium in hPSC-Derived Cardiomyocytes

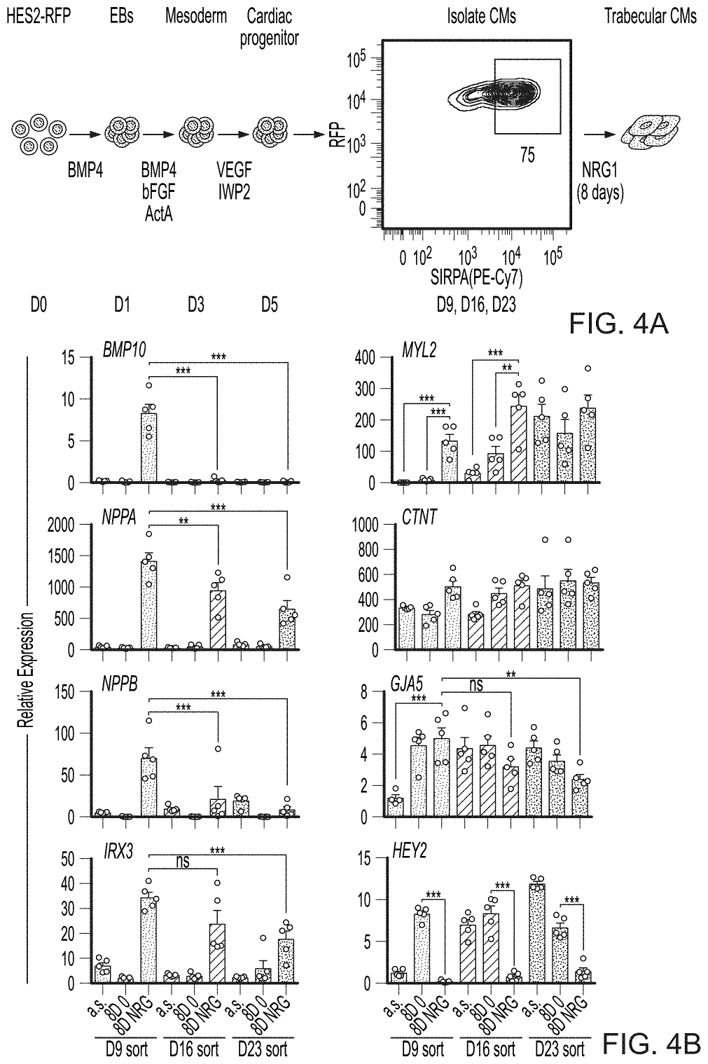

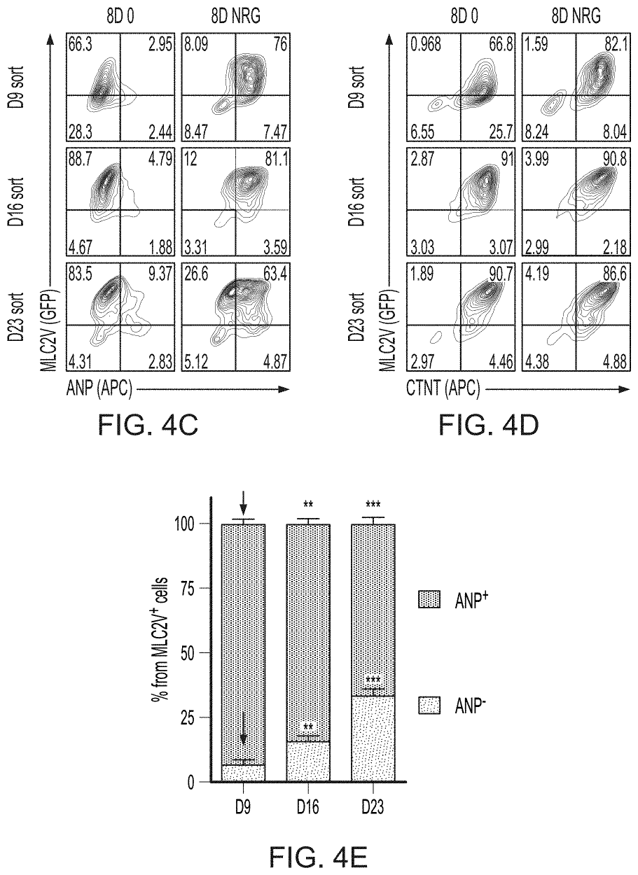

If the NKX2-5+ CD31+ cells represent the equivalent of endocardium, they should be able to induce a trabecular fate in target cardiomyocytes, thereby replicating the interaction that takes place in the early heart tube. As specification of the trabecular fate is mediated by NRG-ERBB signaling in the developing heart tube, the cardiac cells generated with the protocol described herein were tested to determine if they were responsive to this pathway. For this analysis, SIRPA+ cardiomyocyte populations (Dubois et al., 2011, Nat. Biotechnol., 29(11):1011-8) isolated at days 9, 16 and 23 of differentiation were treated with NRG1 (FIG. 4A) for 8 days and then analyzed by RT-qPCR for markers indicative of trabecular cardiomyocytes including BMP10, NPPA, NPPB and IRX3 (Christoffels et al., 2000, Dev. Biol., 224(2):263-74; Chen et al., 2004, Development, 131(9):2219-31; Koibuchi and Chin, 2007, Circ. Res., 100(6):850-5; Miquerol et al., 2010, Circ. Res., 107(1):153-61; Zhang et al., 2011, PNAS USA, 108(33):13576-81; Kim et al., 2012, Circ. Res., 110(11):1513-24; Sergeeva and Christoffels, 2013, Biochim. Biophys. Acta, 1832(12):2403-13; Sergeeva et al., 2014, Cardiovasc. Res., 101(1):78-86). As shown in FIG. 4B, day 9 treated cells expressed higher levels of BMP10, NPPA and NPPB than the later populations. The differences in the levels of IRX3 expression were only detected in the day 23 treated population. The most striking differences were observed with BMP10, as the day 9-derived population was the only one that expressed levels above those found in the untreated cells. Culture in the presence of NRG1 significantly reduced the levels of expression of the compact marker HEY2 (Koibuchi and Chin, 2007, Circ. Res., 100(6):850-5) in cells derived from all stages of differentiation, suggesting that signaling through this pathway is inhibitory to the development of compact cardiomyocytes. All treated population expressed similar levels of MYL2 and CTNT, reflective of the fact that they represent ventricular cardiomyocytes. GJA5, a distinguishing marker of the trabecular lineage in the early embryo was expressed in all populations, indicating that its expression was not upregulated in response to NRG1 in the hPSC-derived cardiomyocyte population.

The treated populations were evaluated by flow cytometric analyses to determine the proportion of MLCV+ cells that co-express the trabecular marker, atrial natriuretic peptide (ANP; encoded by NPPA). These analyses showed that the day 9-derived population contained the highest proportion of ANP+ cells, with an average of 90% of the cells expressing this marker (FIGS. 4C-4E). The populations generated from the later stage cells contained a lower proportion of these cells, a finding consistent with the molecular analyses, which indicated that the trabecular fate is most efficiently induced from SIRPA+ cells isolated at day 9 of differentiation. All populations contained a high frequency of MLC2V+ cells, demonstrating they are ventricular cardiomyocytes (FIGS. 4C, 4D). Culture in the presence of NRG1 promoted the generation of MLC2V+ cells from the day 9 cells, indicating that signaling through this pathway can enhance ventricular differentiation. Collectively, these findings show that day 9 cardiomyocytes represent an appropriate target population for testing the potential of the hPSC-derived endocardial-like cells to induce a trabecular fate.

### Example 14—NKX2-5+ CD31+ Endocardial-Like Cells Induce Markers of Trabecular Myocardium in hPSC-Derived Cardiomyocytes

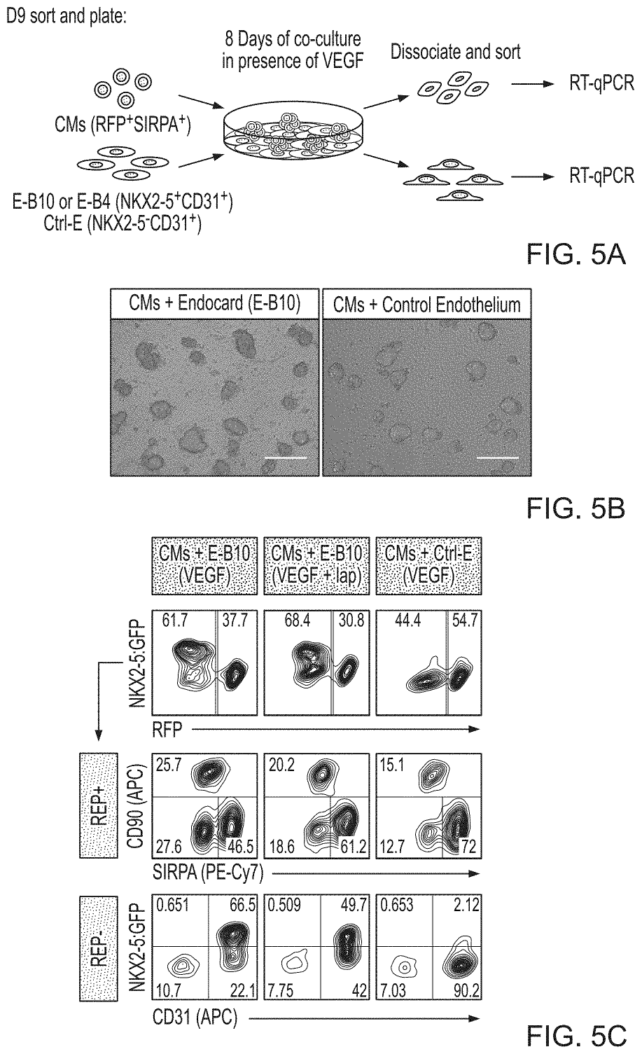

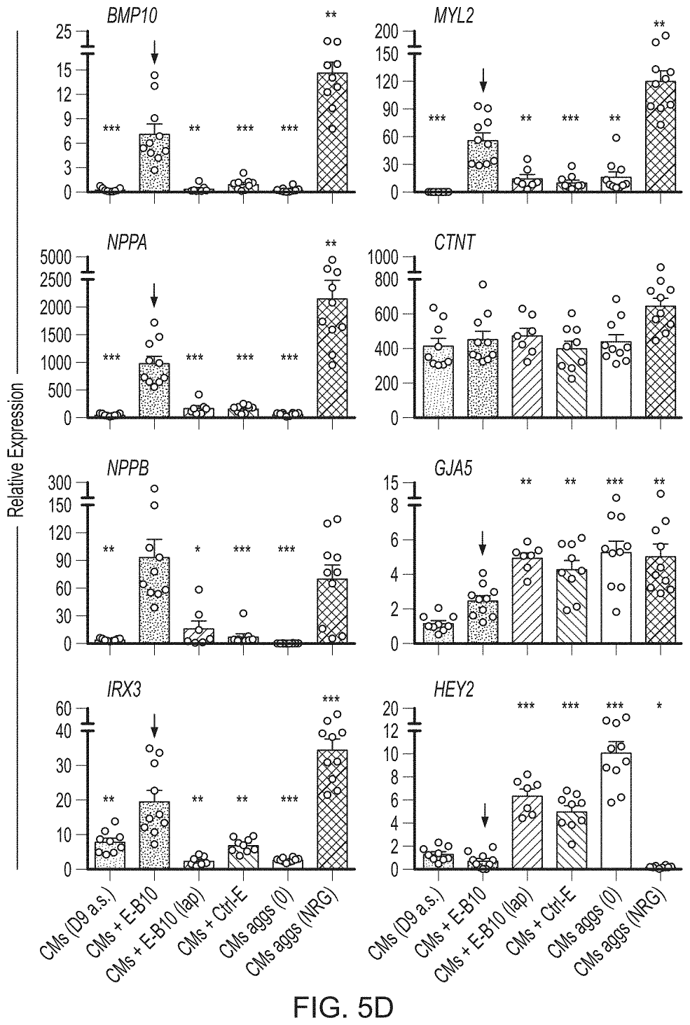

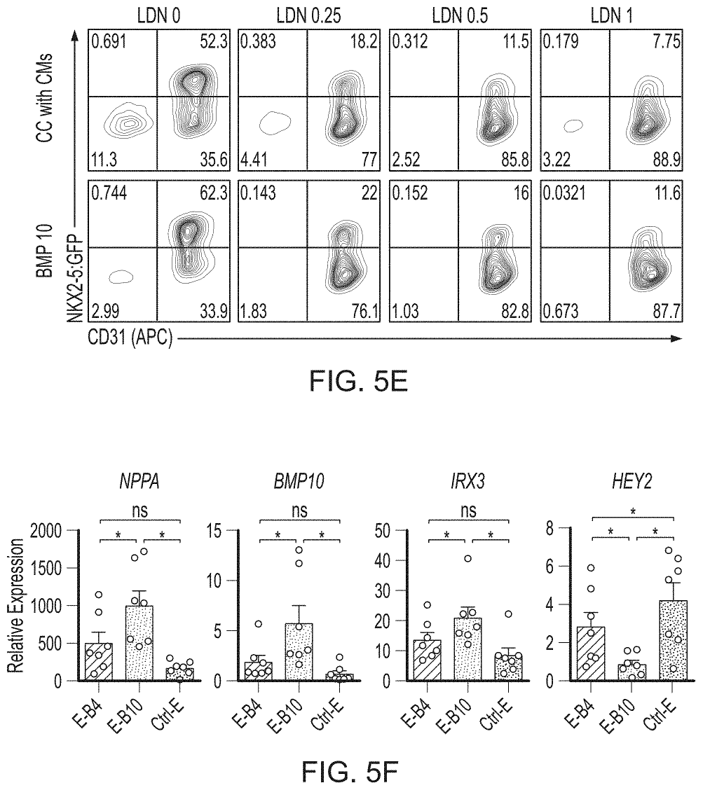

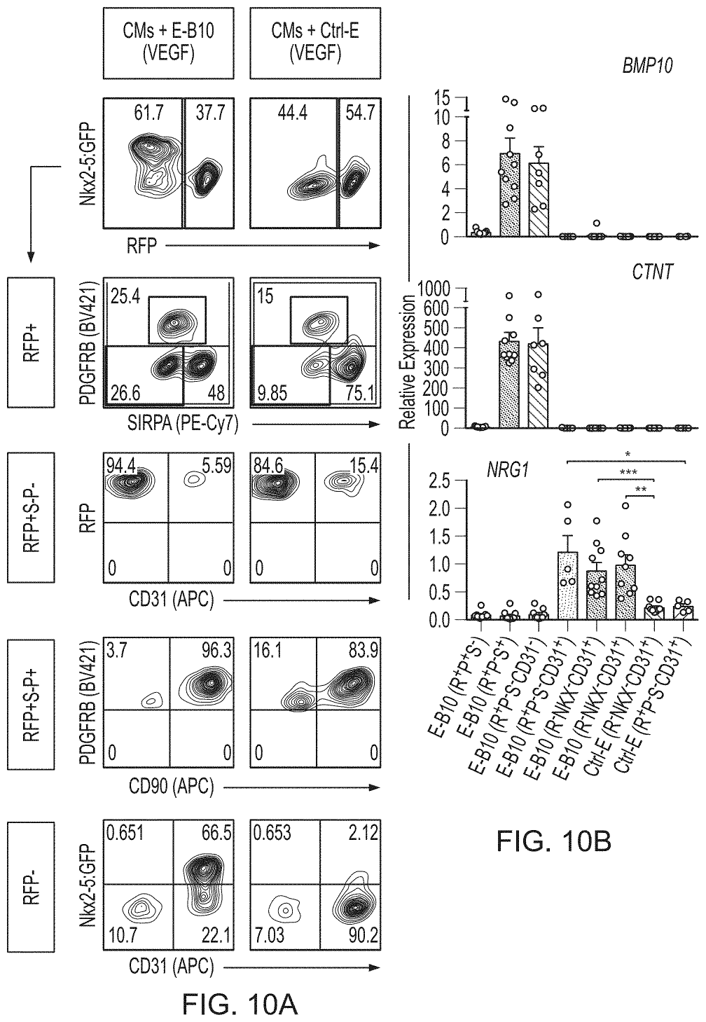

To determine if the NKX2-5+ CD31+ endocardial-like cells have the potential to induce a trabecular fate in the cardiomyocyte target population, day 9 SIRPA+ cardiomyocytes generated from a HES2:RFP hPSC line were mixed with day 9 NKX2-5+ CD31+ cells produced from the HES3:NKX2-5:GFP line and cultured them together in a monolayer format in the presence of VEGFA (FIG. 5A). During co-culture, the NKX2-5+ CD31+ cells segregate with cardiomyocytes, forming aggregates on top of the endothelial cells (FIG. 5B). In addition to the endocardial-like cells, control NKX2-5-CD31+ endothelial cells also were cultured with the cardiomyocytes. As a positive control for specification of the trabecular fate, the cardiomyocytes were cultured as aggregates in the absence of endothelial cells and in the presence of NRG1. Following eight days, the cultured populations were dissociated to single cells and segregated into different fractions by FACS (FIG. 5C, FIG. 10A). The endocardial/endothelial cells were isolated as a RFP− CD31+ population, whereas the cardiomyocytes were identified as RFP+ SIRPA+ cells. There was significant outgrowth of a SIRPA− population from the RFP+ SIRPA+ fraction during the co-culture. This population consisted of CD90+ PDGFRB+ SIRPA− mesenchymal-like cells, CD90− PDGFRB− SIRPA− CD31− cardiomyocytes and CD90+ PDGFRB− SIRPA− CD31+ endothelial cells (FIG. 5C, FIG. 10A). SIRPA− cells also were generated following co-culture with the control endothelial cells.

RT-qPCR analyses revealed that expression of the markers of trabecular myocardium (BMP10, NPPA, NPPB, IRX3) were significantly upregulated in the RFP+ SIRPA+ cardiomyocytes co-cultured with the NKX2-5+ CD31+ endocardial-like cells in a pattern similar to that observed in the aggregates treated with NRG1. By contrast, expression of these genes was not upregulated in the cardiomyocytes cultured with the control endothelial cells or in the cardiomyocyte aggregates cultured in the absence of NRG1. The expression pattern of the compact marker HEY2 was opposite to that of the trabecular markers, as it was upregulated in the cardiomyocytes cultured with the control endothelial cells, but not in the cells cultured with the endocardial-like cells. All populations expressed comparable levels of CTNT. MYL2 expression was upregulated in the cardiomyocytes co-cultured with the NKX2-5+ CD31+ endocardial-like cells and in the cardiomyocyte aggregates induced with NRG1, indicative of ventricular maturation. As observed in the above analyses, expression of GJA5 did not segregate with the patterns of the other trabecular markers.

Induction of the trabecular fate by the NKX2-5+ CD31+ endocardial-like cells was inhibited by the addition of the ERBB2 inhibitor lapatinib (lap), indicating that the effect is mediated by NRG-ERBB signaling. This interpretation is further supported by the finding that the NKX2-5+ CD31+ cells isolated from the co-culture population express NRG1 (FIG. 10B). In addition to the NKX2-5+ CD31+, the derivative NKX2-5− CD31+ cells as well as the CD31+ cells generated from the SIRPA+ RFP+ cardiomyocyte enriched population also expressed NRG1. In contrast, the control CD31+ cells did not upregulate NRG1 expression (FIG. 10B).

The majority of the NKX2-5+ CD31+ endocardial-like cells from the co-culture population maintained expression of NKX2-5 similar to those cultured with exogenous BMP10 (FIG. 5E). This expression is likely mediated by BMP10, which is expressed by cardiomyocytes as they acquire a trabecular fate following co-culture with the endocardial cells (FIGS. 5D and 10B). A role for BMP signaling in maintaining NKX2-5 expression is supported by the finding that addition of the BMP inhibitor, LDN193189, resulted in a partial downregulation of NKX2-5 expression in endocardial cells isolated from the co-cultures as well as in cells treated with BMP10 (FIG. 5E).

Taken together, the findings from these co-culture experiments demonstrate that the NKX2-5+ CD31+ endocardial-like cells can induce a trabecular fate in the cardiomyocyte target population and that this induction is mediated through NRG-ERBB signaling. The ability to induce a trabecular fate appears to be specific to the endocardial like cells, as the control endothelial cells do not display this activity. Given this specificity, the upregulation of trabecular genes can be used as an in vitro developmental assay to assess endocardial potential of any endothelial population. To test this, the potential of BMP4 induced NKX2-5+ CD31+ cells to those induced with BMP10 was compared. As shown in FIG. 5F, the endocardial cells generated with BMP4 failed to induce the expression of the trabecular genes above the levels observed in the cardiomyocytes cultured with the control endothelial cells. Co-culture with the BMP10-induced cells led to a significant up regulation of the trabecular genes and a downregulation of HEY2, indicating specification of a trabecular fate. The differences in the potential of these populations are consistent with differences in the expression levels of NRG1 between them (FIG. 3C).

### Example 15—Generation of NKX2-5+ CD31+ Endocardial Cells From Different Mesoderm Populations

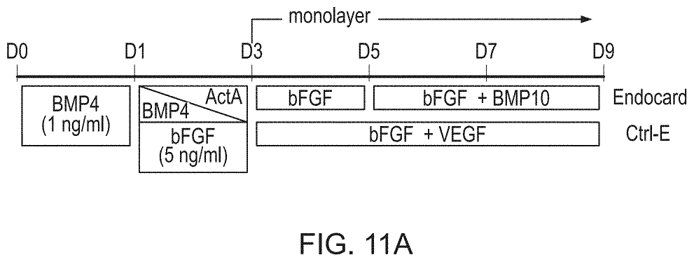

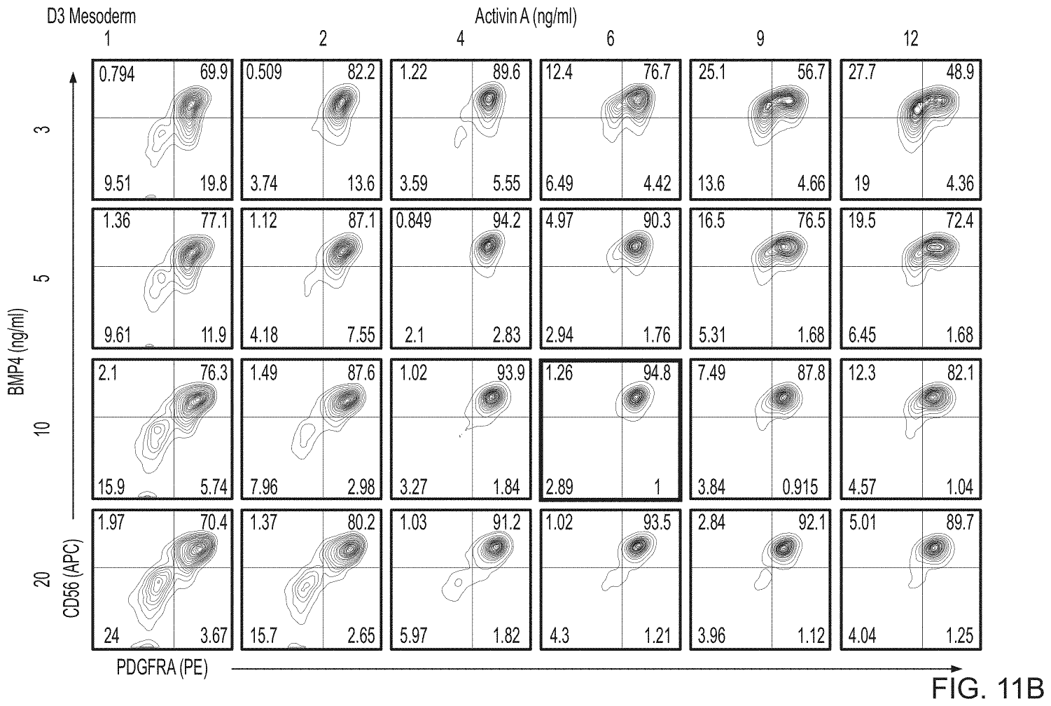

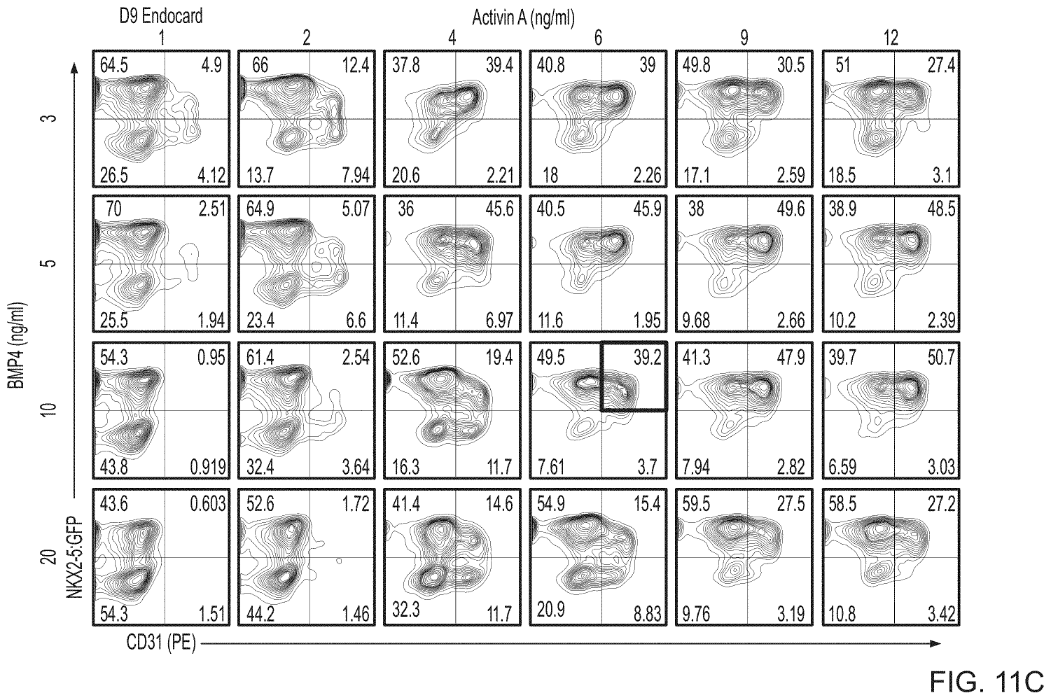

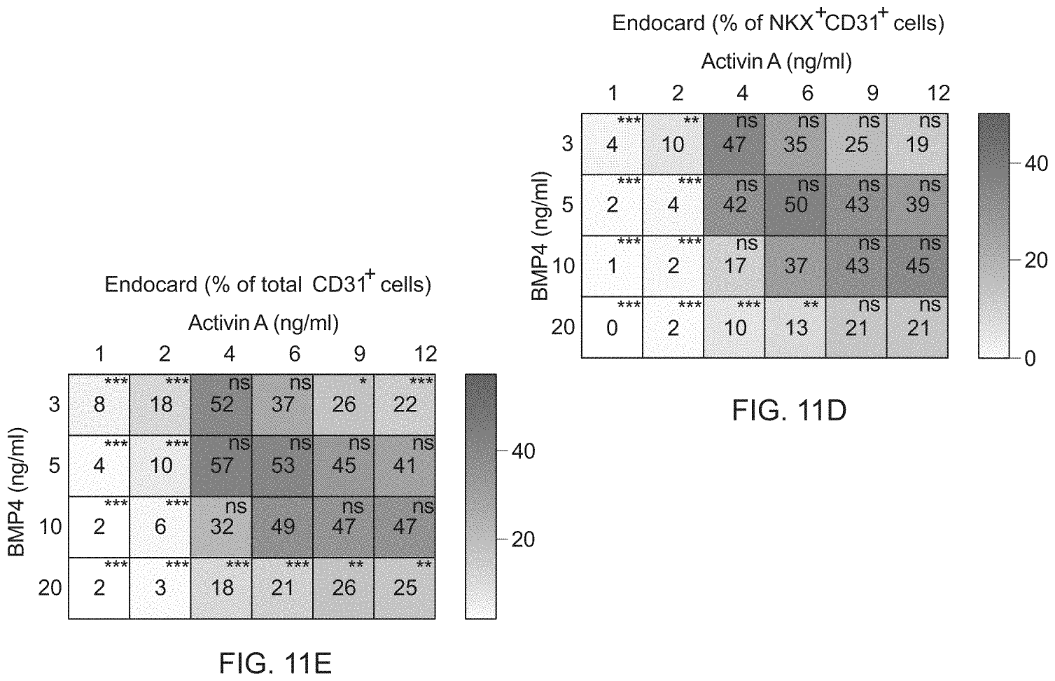

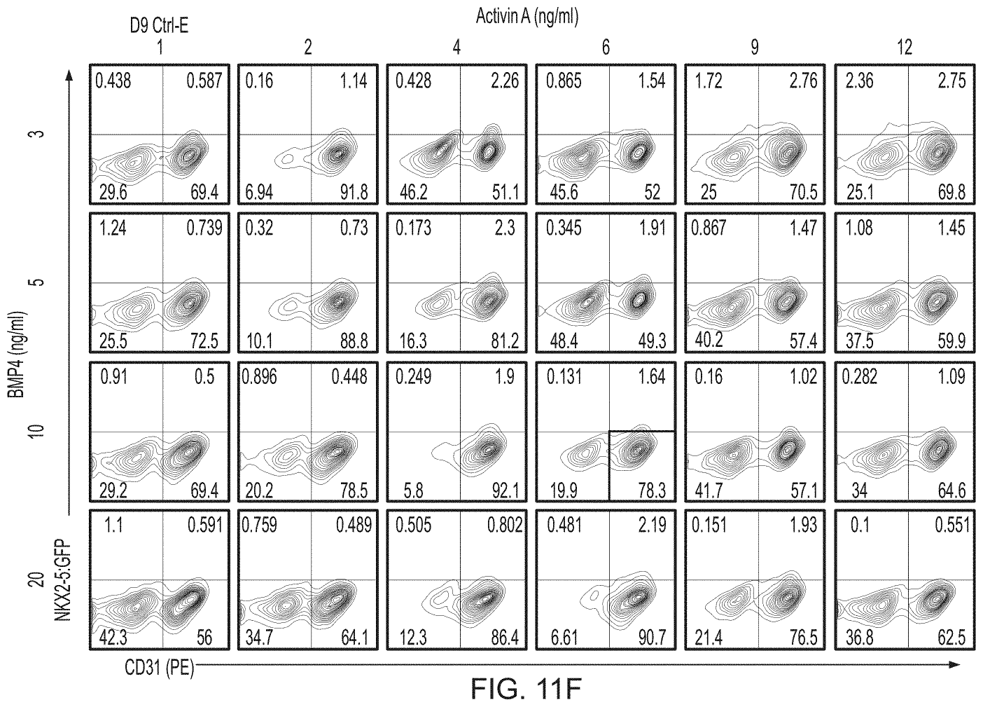

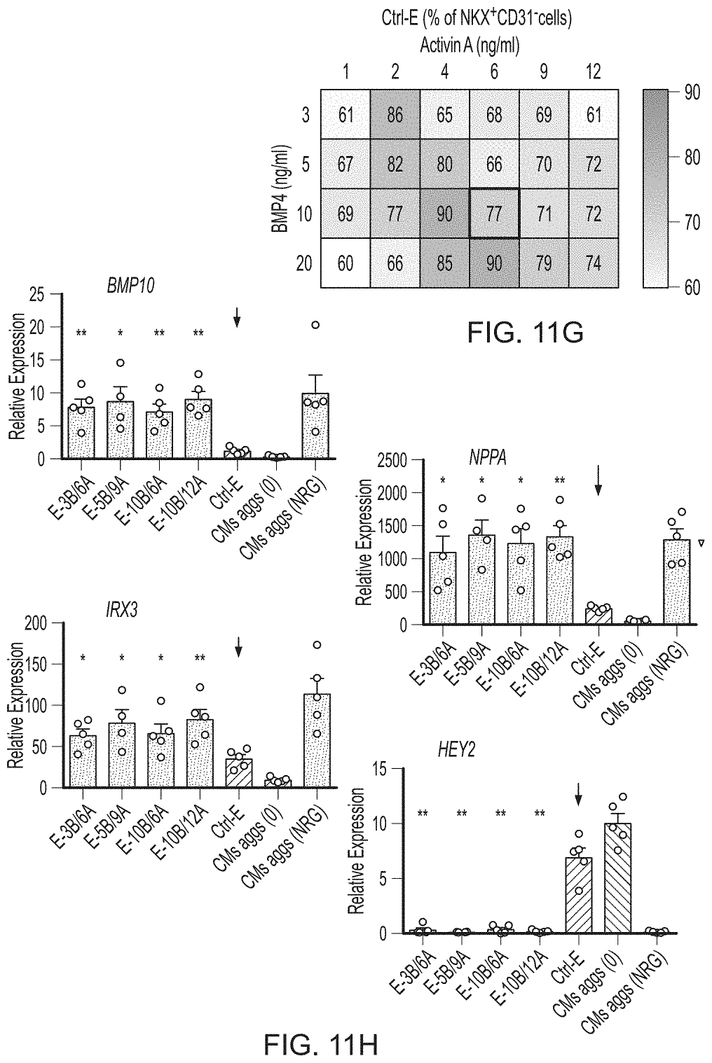

Ventricular and atrial cardiomyocytes develop from distinct mesoderm subpopulations that are induced with different concentrations of BMP4 and Activin A. The NKX2-5+ CD31+ endocardial cells characterized in the above studies were generated from mesoderm induced with a ventricular protocol (10 ng/ml of BMP4, 5 ng/ml of bFGF and 6 ng/ml of ActA, referred to as “10B/6A”). To determine if the endocardial fate is restricted to this mesoderm and to establish the overall importance of the mesoderm induction step in the generation of these cells, mesoderm were induced with a range of concentrations of BMP4 (3-20 ng/ml) and Activin A (1-12 ng/ml) and then the cells were cultured in either endocardial or control endothelial conditions (FIG. 11A, 11B). All factor combinations induced CD56+ PDGFRA+ mesoderm, although the efficiency varied with the high concentration of activin and low concentration of BMP4 being the least efficient (FIG. 11B). Flow cytometric analyses of the day 9 populations revealed that only a subset of mesoderm populations generated NKX2-5+ CD31+ cells (FIG. 11C). Populations induced with activin concentrations of greater than 4 ng/ml and BMP concentrations of less than 20 ng/ml gave rise to the highest proportion of NKX2-5+ CD31+ cells. Lower amounts of activin (1-2 ng/ml) induced mesoderm that gave rise to significantly smaller NKX2-5+ CD31+ populations at all concentrations of BMP4 tested. The lack of NKX2-5+ CD31+ potential was most pronounced in the groups induced with the highest amounts of BMP4 (10-20 ng/ml). A comparison of the endocardial potential of the different mesoderm populations is summarized in FIG. 11D. Analyses of these patterns revealed that the efficiency of NKX2-5+ CD31+ cell development following BMP10 specification correlated well with the efficiency of total CD31+ cell development (FIG. 11D, 11E). In contrast to the restricted patterns of NKX2-5+ CD31+ endocardial cell development, populations induced with all combinations of activin and BMP generated high proportions (average >60% of total population) of CD31+ control endothelial cells (FIGS. 11F, 11G). Taken together, these findings suggest that only a subset of the mesodermal populations produced can generate NKX2-5+ CD31+ cells.

Put another way, different subsets of mesoderm were produced by culturing hESCs in the presence of different amounts of BMP4 and Activin A. All the mesodermal populations that were generated with the range of concentrations of Activin A and BMP4 that were tested were capable of producing control endothelial cells upon culturing them in the presence of VEGFA and bFGF, but only a small subset of amounts of BMP4 and Activin A from the range that were tested produced mesodermal populations that were capable of generating endocardial cells upon culturing them in the presence of bFGF and BMP10.

To further characterize cells induced under different conditions, NKX2-5+ CD31+ cells generated from 3 different inductions (“3B/6A”, “5B/9A”, or “10B/12A”) were analyzed and compared to cells generated under our standard conditions (10B/6A). The different NKX2-5+ CD31+ cells were evaluated for their ability to induce a trabecular fate in day 9 target cardiomyocytes and for levels of NRG1 expression. All 3 NKX2-5+ CD31+ populations were able to induce a trabecular profile similar to that induced by the 10B/6A cells (FIG. 11H). As observed in previous experiments described herein, the cardiomyocytes co-cultured with the control endothelial cells did not express the trabecular markers, but rather expressed HEY2, indicative of a compact fate. Consistent with these findings, the RT-qPCR analyses showed that the different NKX2-5+ CD31+ populations expressed NRG1 prior to and following co-culture at levels similar to those in the cells generated from the 10B/6A mesoderm and significantly higher than in the control endothelial cells (FIG. 11I).

### Example 16—Generation of Endocardial-Like Cells From HES2-RFP hESCs

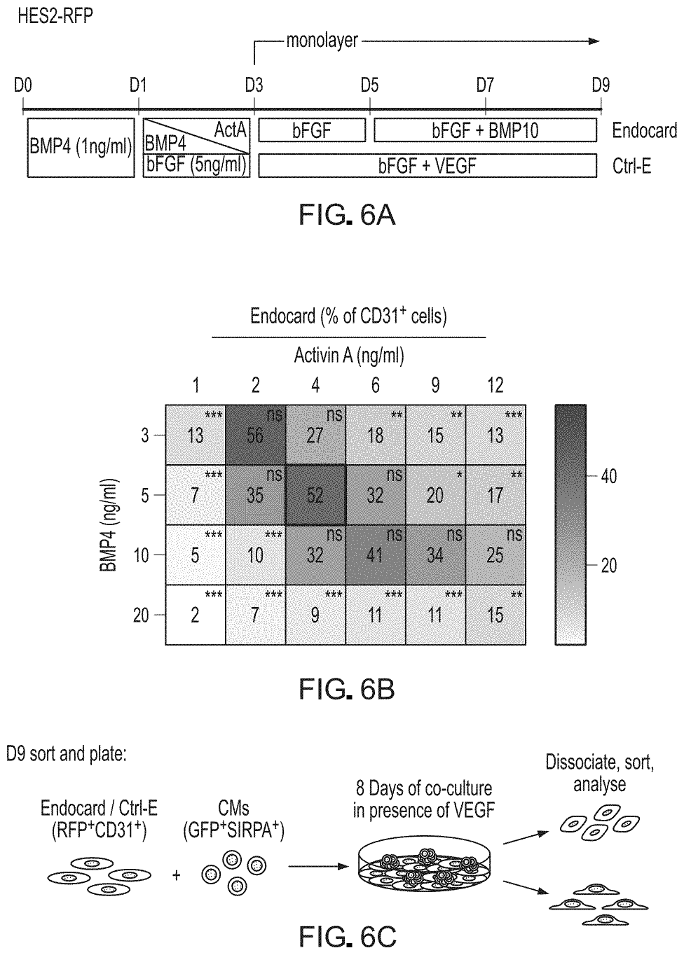

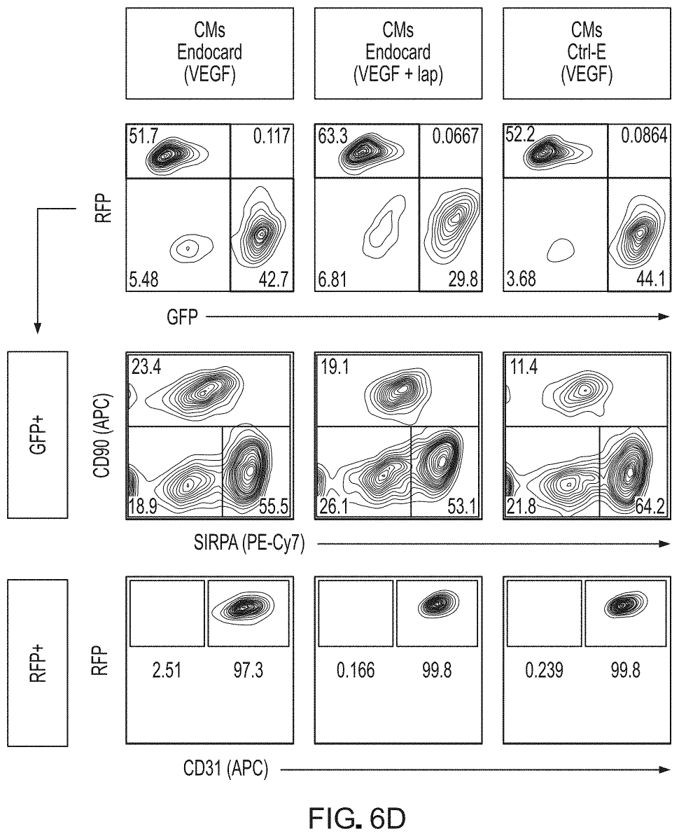

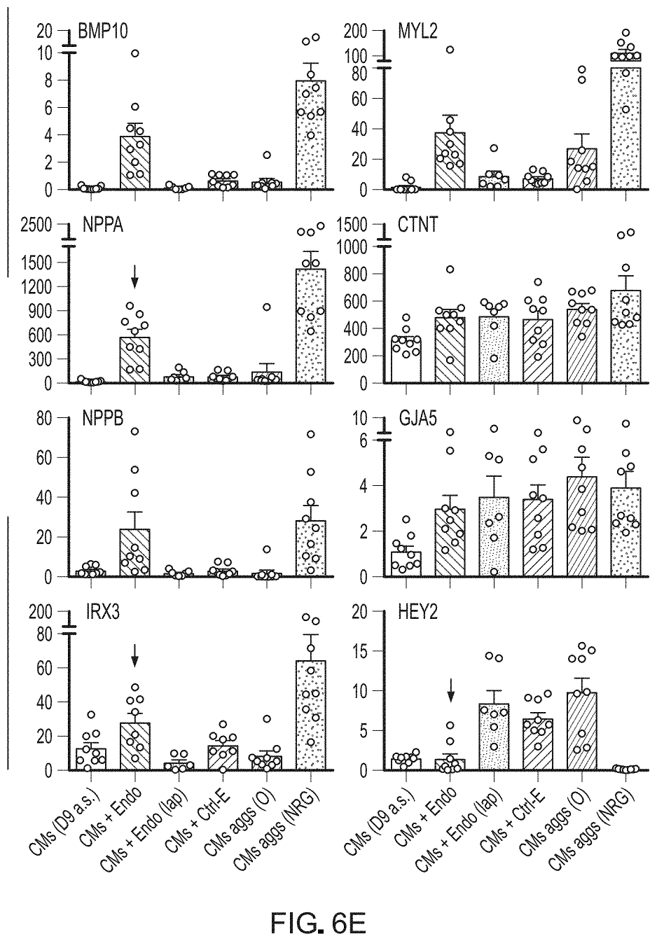

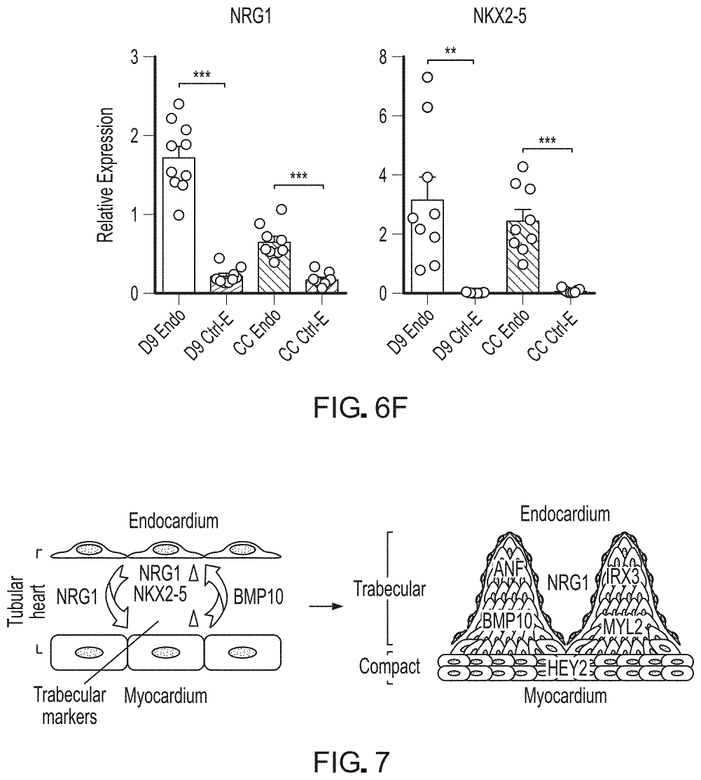

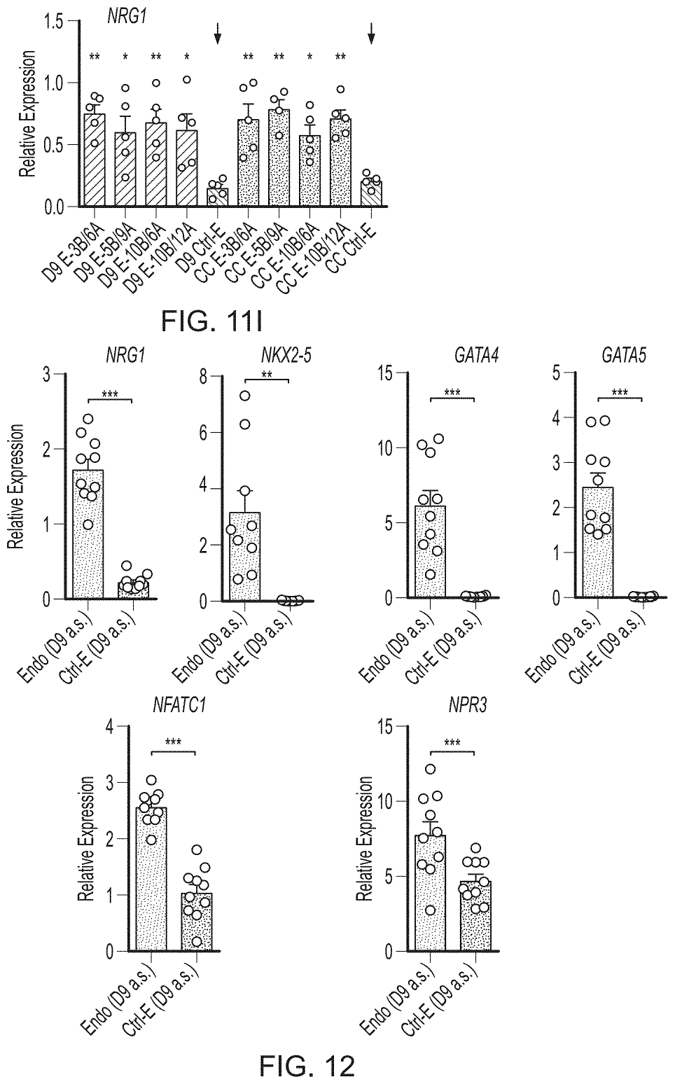

As the development of NKX2-5+ CD31+ cells correlated with the development of total CD31+ cells under endocardial conditions, this readout was used to optimize induction strategies for the generation of endocardial-like cells from a second hPSC line that did not contain a NKX2-5 reporter. These cells were engineered to constitutively expresses RFP (HES2-RFP) (FIG. 6A). As shown in FIG. 6B, CD31+ cells could be generated from day 3 populations induced with a range of BMP4 and activin concentrations. From these, the mesoderm induced with 5 ng/ml of BMP4 and 4 ng/ml of Activin A (5B4A mesoderm) was selected for the generation of CD31+ endocardial-like cells and CD31+ control endothelial cells. Molecular analyses showed that these HES2− RFP-derived CD31+ endocardial-like cells expressed significantly higher levels of NRG1, NKX2-5, GATA4,GATA5,NFATC1 and NPR3 than CD31+ control endothelial cells (FIG. 12).

To measure endocardial potential (induction of trabecular fate), both populations were isolated from day 9 EBs and co-cultured with SIRPA+ cardiomyocytes generated from a hPSC line that expresses GFP (HES2-GFP) (FIGS. 6C, 6D). Analyses of the isolated SIRPA+ CD90− GFP+ cardiomyocytes following co-culture (FIGS. 6D, 6E) revealed that the RFP+ CD31+ endocardial-like cells generated from the 5B4A mesoderm induced the expression of trabecular myocardium markers and suppressed the upregulation of HEY2 in this target population. In contrast, the RFP+ CD31+ control endothelial cells failed to induce the trabecular fate. The induction of these markers was inhibited by the addition of lapatinib to the cultures, indicating that the effect is mediated by NRG-ERBB as observed with the NKX2-5+ CD31+ cells (FIG. 6E). The observation that the RFP+ CD31+ endocardial-like cells express higher levels of NRG1 and NKX2-5 than the RFP+ CD31+ control endothelial cells, prior to and following co-culture supports this interpretation (FIG. 6F).

Collectively, the findings from this set of experiments clearly demonstrate that it is possible to generate endocardial-like cells from non-targeted (NKX2-5) hPSCs and that expression of CD31 can be used to monitor the development of this population and to isolate it for functional analyses.

### Example 17—Generation of VIC-Like Cells From hPSC-Derived Endocardial-Like Cells

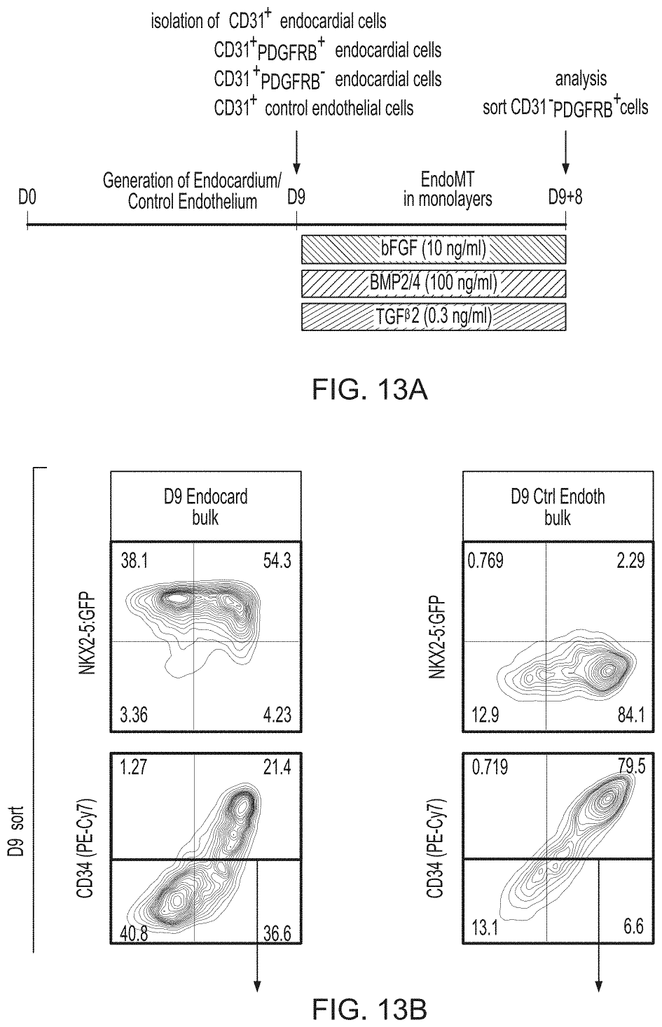

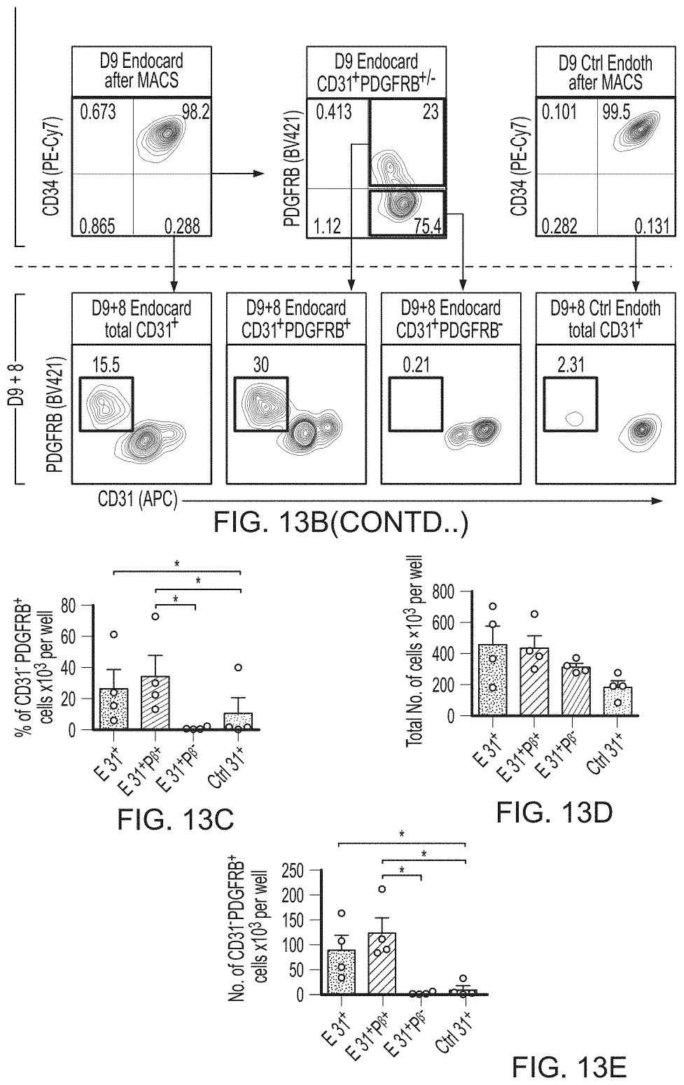

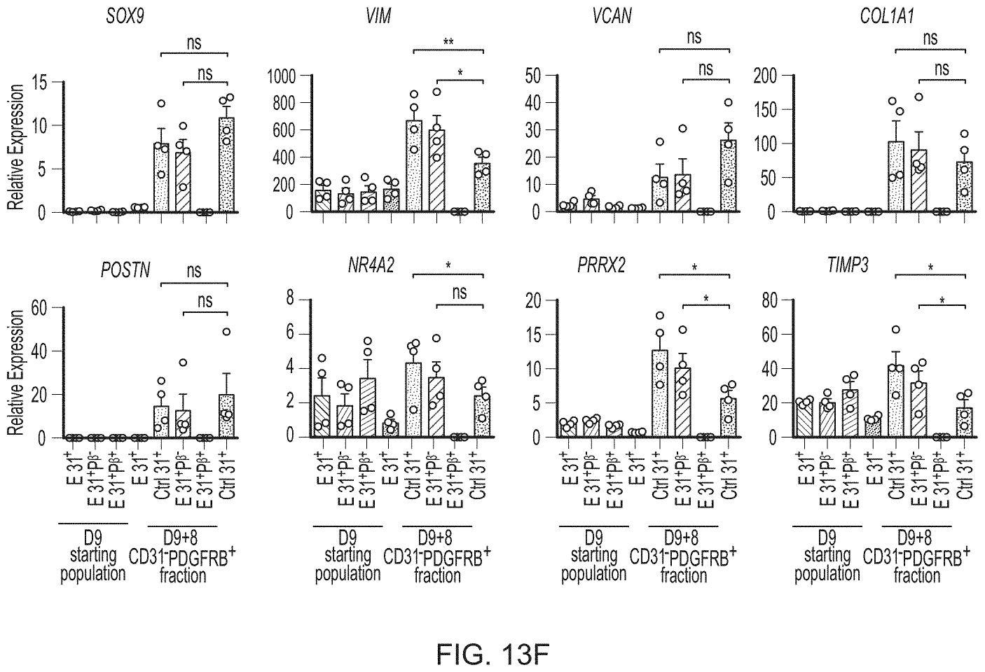

To assess the potential of hPSC-derived endocardial-like cells to undergo endothelial-to-mesenchymal transition, the endothelial fractions of hPSC-derived endocardial-like cells and control endothelial cells were isolated at day 9 using magnetic-activated cell sorting (MACS) (FIG. 13A). Day 9 endocardial-like cells and control endothelial cells were found to co-express CD34 with CD31 (FIG. 13B), which allowed for effective purification of the endothelial compartment using CD34-specific magnetic beads This strategy allowed us to routinely isolate populations that consist of more than 95% CD31+ cells and greater than 85% NKX2-5+ CD31+ cells (FIG. 13B) and to scale up endocardial cell production for further experiments. The endothelial fraction of day 9 endocardial-like cells was further divided into CD31+ PDGFRb+ and CD31+ PDGFRb− fractions by FACS (FIG. 13A, 13B). These fractions were subsequently cultured for 8 days in the presence of 100 ng/mL of BMP2/4, 10 ng/mL of bFGF and 0.3 ng/mL of TGFb2 (FIG. 13A), known inducers of endothelial-to-mesenchymal transition in vivo. Following 8 days of culture, there was a robust generation of CD31− PDGFRb+ cells (i.e. mesenchymal cells) from the CD31+ PDGFRb+ endocardial subpopulation, but not from the CD31+ PDGFRb− cells (FIG. 13B-13E). Control endothelial cells, cultured under the same conditions, also yielded a population of CD31− PDGFRb+ cells, albeit much smaller than that generated from the CD31+ PDGFRb+ endocardial-like cells (FIG. 13B-13E). qPCR analysis demonstrated similar upregulation of general mesenchymal markers (SOX9, VCAN, COL1A1 and POSTN) in the CD31− PDGFRb+ cells derived from both the CD31+ PDGFRb+ endocardial-like cells and control endothelial cells, and higher levels of a general mesenchymal marker VIM in CD31+ PDGFRb+ endocardial-like cells compared to control endothelial cells (FIG. 13F). Furthermore, there was an up-regulation of the VIC-specific markers NR4A2, PRRX2 and TIMP3 in the CD31− PDGFRb+ cells derived from the CD31+ PDGFRb+ endocardial-like cells compared with control endothelial cells (FIG. 13F).

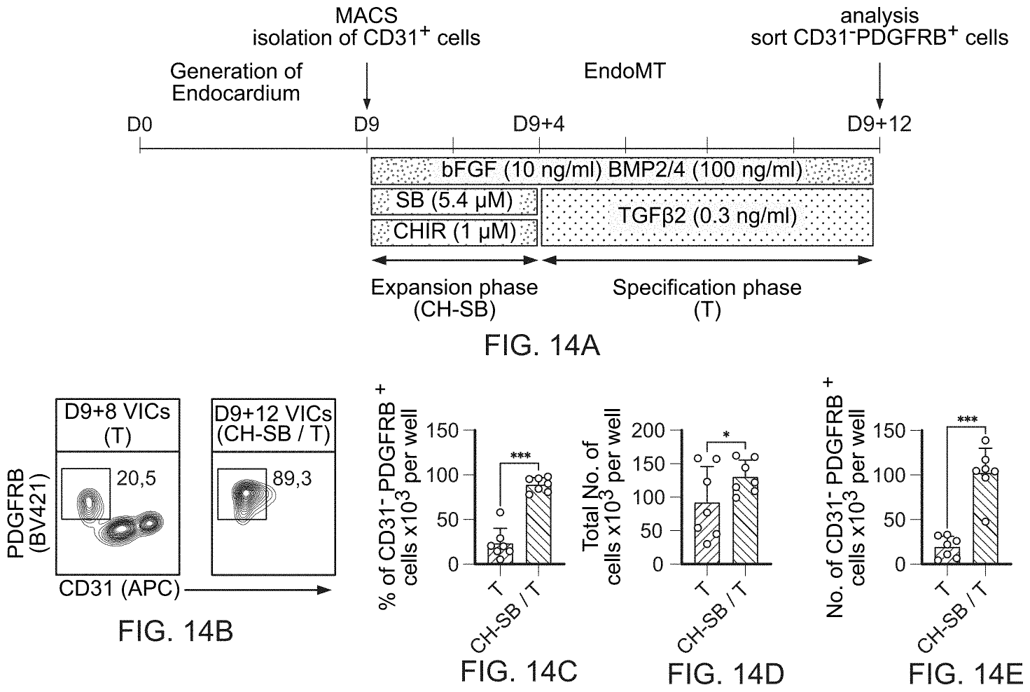

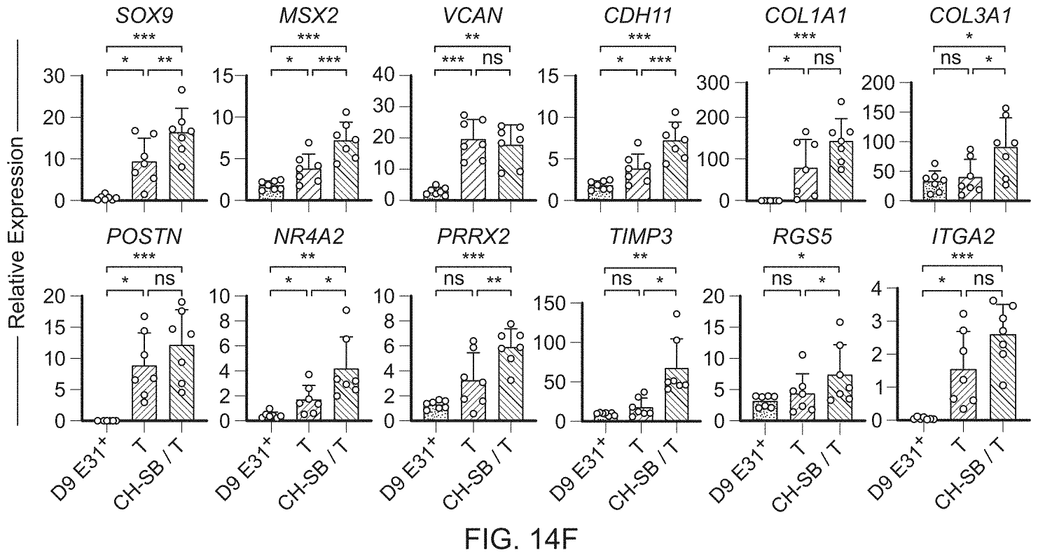

In order to enrich for CD31− PDGFRb+ VIC-like cells, activation of the Wnt signaling pathway with 1 μM CHIR99021 was tested prior to specification of the VIC fate with BMP2/4, bFGF and TGFb2. Day 9 CD31+ MACS-sorted endocardial-like cells were cultured in the presence of 1 μM CHIR99021, 10 ng/mL of bFGF and 100 ng/mL of BMP2/4 as well as 5.4 μM of the TGFbeta inhibitor SB-431542 to stimulate cell proliferation for four days (FIG. 14A). This expansion phase was followed by the previously used specification phase with BMP2/4, bFGF and TGFb2 for eight days. Application of CHIR, SB, bFGF and BMP2/4 led to a dramatic increase of the purity of the CD31− PDGFRb+ VIC-like population (from 20% to 90%, FIG. 14B) as well as cell numbers (5-fold, FIG. 14C-E). Addition of the CHIR/SB phase did not affect the expression of general mesenchymal markers (VCAN, COL1A1, COL3A1 and POSTN) and increased the expression of VIC-specific markers NR4A2, PRRX2, TIMP3, and RGS5 (FIG. 14F).

Collectively, the findings from this set of experiments demonstrate that a subpopulation of hPSC-derived endocardial-like cells, marked by the expression of PDGFRb, can undergo an endothelial-to-mesenchymal transition, and do so more efficiently than control endothelial cells. Furthermore, when compared with mesenchymal cells derived from control endothelial cells, these endocardial-derived mesenchymal cells looked more like VICs, based on signature gene expression, than the mesenchymal cells derived from control endothelial cells.

Key resources table

| | | |
|---|---|---|
| Reagent or Resource | Source | Identifier |

Antibodies

| | | |
|---|---|---|
| Mouse monoclonal to | Biolegend | Cat. # 323808; RRID: |
| SIRPα (clone SE5A5), PeCy7 | | AB_1236443 |
| conjugated | | |
| Mouse monoclonal to CD90 | BD Pharmingen | Cat. # 559869; RRID: |
| (clone 5E10), APC conjugated | | AB_398677 |
| Mouse monoclonal to CD31 | BD Pharmingen | Cat. # 555446; RRID: |
| (clone WM59), PE conjugated | | AB_395839 |
| Mouse monoclonal to CD31 | BD Pharmingen | Cat. # 555445; RRID: |
| (clone WM59), FITC conjugated | | AB_395838 |
| Mouse monoclonal to CD31 | BD Pharmingen | Cat. # 561654; RRID: |
| (clone WM59), Alexa Fluor 647 | | AB_10896969 |
| conjugated | | |
| Mouse monoclonal to CD34 | ThermoFisher, | Cat. # 25-0349-42; |
| (clone 4H11), PeCy7 conjugated | eBioscience | RRID: AB_1963576 |
| Mouse monoclonal to CD36 | Biolegend | Cat. # 336208 RRID; |
| (clone 5-271), APC conjugated | | AB_1279224 |
| Mouse monoclonal to CD140b | BD Horizon | Cat. # 564124; RRID: |
| (PDGFRβ) (clone 28D4), BV421 | | AB_2738609 |
| conjugated | | |
| Mouse monoclonal to CD105 | ThermoFisher, | Cat. # 17-1057-42; |
| (Endoglin) (clone SN6), APC | eBioscience | RRID: AB_1582211 |
| conjugated | | |
| Mouse monoclonal to PDGFRα | BD Pharmingen | Cat. # 556002; RRID: |
| (clone αR1), PE conjugated | | AB_396286 |
| Mouse monoclonal to CD56 | BD Pharmingen | Cat. # 555518; RRID: |
| (clone B159), APC conjugated | | AB_398601 |
| Mouse monoclonal anti human | Dako | Cat. # M0823; RRID: |
| CD31 (clone JC70A), non- | | AB_2114471 |
| conjugated | | |
| Mouse monoclonal to CTNT | ThermoFisher | Cat. # MS-295-P; RRID: |
| (clone 13-11), non-conjugated | | AB_11000742 |
| Rabbit polyclonal to MLC2V | Abcam | Cat. # ab79935; RRID: |
| (clone 13-11), non-conjugated | | AB_1952220 |
| Mouse monoclonal to ANP | Abcam | Cat. # ab2093; RRID: |
| (M622709), non-conjugated | | AB_302831 |
| Rabbit monoclonal to NKX2-5 | Cell Signaling | Cat. # 8792S; RRID: |
| (E1Y8H), non-conjugated | | AB_2797667 |
| Rabbit polyclonal to NFAT2, | Abcam | Cat. # ab25916; RRID: |
| non-conjugated | | AB_448901 |
| Rabbit polyclonal to GATA4, | Abcam | Cat. # ab84593; RRID: |
| non-conjugated | | AB_10670538 |
| Mouse monoclonal anti-human | Biolegend | Cat. # 744847; RRID |
| LDLR (clone c7), | | |
| BrilliantViolet421 conjugated | | |
| Goat anti-mouse IgG (H + L), | BD Pharmingen | Cat. # 550826; RRID: |
| APC conjugated | | AB_398465 |
| Donkey anti-rabbit IgG (H + L), | ThermoFisher | Cat. # A21206; RRID: |
| AlexaFluor488 conjugated | | AB_2535792 |
| Goat anti-GFP, non-conjugated | Rockland | Cat. # 600-101-215; |
| | | RRID: AB_218182 |
| Donkey anti-goat IgG (H + L), | ThermoFisher | Cat. # A11055; RRID: |
| AlexaFluor488 conjugated | | AB_2534102 |
| Donkey anti-rabbit IgG (H + L), | ThermoFisher | Cat. # A31572; RRID: |
| AlexaFluor555 conjugated | | AB_162543 |
| Donkey anti-mouse IgG (H + L), | ThermoFisher | Cat. # A31571; RRID: |
| AlexaFluor647 conjugated | | AB_162542 |

Chemicals, Peptides, and Recombinant Proteins

| | | |
|---|---|---|
| Penicillin/streptomycin | ThermoFisher | Cat. # 15070063 |
| L-glutamine | ThermoFisher | Cat. # 25030081 |
| Non-essential amino acids | ThermoFisher | Cat. # 11140-050 |
| Transferrin | ROCHE | Cat. # 10652202001 |
| Ascorbic acid | Sigma | Cat. # A4544-100G |
| Monothioglycerol | Sigma | Cat. # M-6145-25 ml |
| β-Mercaptoethanol | ThermoFisher | Cat. # 21985-023 |
| ROCK inhibitor Y-27632 | Tocris | Cat. # 1254 |
| Recombinant human BMP2 | R&D | Cat. # 355-BM |
| Recombinant human BMP4 | R&D | Cat. # 314-BP |
| Recombinant human ActivinA | R&D | Cat. # 338-AC |
| Recombinant human TGFβ2 | R&D | Cat. # 302-B2 |
| Recombinant human bFGF | R&D | Cat. # 233-FB |
| Recombinant human BMP10 | R&D | Cat. # 2926-BP |
| Recombinant human VEGFA | R&D | Cat. # 293-VE |
| Recombinant human VEGFB | R&D | Cat. # 751-VEB-025 |
| Recombinant human NRG1β1 | R&D | Cat. # 396-HP |
| IWP2 (Wnt inhibitor) | Tocris | Cat. # 3533 |
| CHIR 99021 (GSK-3 inhibitor) | Tocris | Cat. # 4423 |
| SB-431542 (TGFbeta inhibitor, | Sigma-Aldrich | Cat. # S4317 |
| SB) | | |
| Lapatinib | LC labs | Cat. # L-4804 |
| LDN 193189 (BMP inhibitor) | Tocris | Cat. # 6053 |
| GW7647 (PPARa agonist) | Sigma | Cat. # G6793 |
| Collagenase type2 | Worthington | Cat. # LS004-176 |
| DAPI | Biotium | Cat. # 40043 |
| Fetal calf serum (FCS) | Wisent | Cat. # 080-150 |
| Bovine serum albumin (BSA) | Sigma | Cat. # A1470 |
| Matrigel, growth factor reduced | Coming | Cat. # 356230 |

Critical Commercial Assays

| | | |
|---|---|---|
| RNAqueous-micro kit with | Ambion | Cat. # AM1931 |
| RNase-free Dnase treatment | | |
| Superscript III Reverse | ThermoFisher | Cat. # 18080044 |
| Transcriptase kit | | |
| QuantiFast SYBR Green PCR kit | QIAGEN | Cat. # 204057 |
| CD34 MicroBead Kit, human | Miltenyi Biotec | Cat. # 130-046-702 |
| MACS cell separation, LS | Miltenyi Biotec | Cat. # 130-042-401 |
| columns | | |

Experimental models: Cell Lines

| | | |
|---|---|---|
| Human ESC: HES3-NKX2- | Gift from Drs. E. | NA |
| 5^{eGFP/w} line | Stanley and A. | |
| | Elefanty, Monash | |
| | University, AU (Elliott, | |
| | Braam et al. 2011) | |
| Human ESC: HES2:RFP line | Generated in Keller lab | NA |
| | from HES2 (ESI | |
| | International | |
| | Singapore) (Irion, | |
| | Luche et al. 2007) | |
| Human ESC: HES2:GFP line | Generated in Keller lab | NA |
| | from HES2 (ESI | |
| | International | |
| | Singapore) | |

Oligonucleotides

| | | |
|---|---|---|
| See Table below for PCR primer | This paper | Table below |
| sequences | | |

Software and Algorithms

| | | |
|---|---|---|
| FlowJo | Tree Star | https://www.flowjo.com |
| FV10-ASW | Olympus | https://www.olympus- |
| | | lifescience.com |
| GraphPad Prism 6 | GraphPad Software | http://www.graphpad.co |
| | | m/scientific- |
| | | software/prism |

Other

| | | |
|---|---|---|
| StemPro-34 media (kit) | ThermoFisher | Cat. # 10639011 |
| DMEM/F12 | Cellgro | Cat. # 10-092-CV |
| KnockOut serum replacement | ThermoFisher | Cat. # 10828028 |
| TrypLE | ThermoFisher | Cat. # 12605010 |
| 96-well clear flat bottom TC- | Falcon | Cat. # 353072 |
| treated culture microplate | | |
| 24-well clear flat bottom TC- | Falcon | Cat. # 353047 |
| treated culture microplate | | |
| 96-well clear flat bottom ultra- | Coming | Cat. # 3474 |
| low attachment microplate | | |
| Petri dishes 60 × 15 mm | VWR | Cat. # 25384-090 |
| Micro cover glasses 12 mm | VWR | Cat. # 89015-725 |
| ProLong diamond antifade | ThermoFisher | Cat. # P36965 |
| mountant | | |

| | | SEQ | | SEQ |
|---|---|---|---|---|
| | | ID | | ID |
| Gene | Forward (5′-3′) | NO | Reverse (5′-3′) | NO: |
| APOD | TGG CAA GAA ACC CTA ATC TCC | 1 | CTG GTC TGT GAC CGT CAT TT | 2 |
| BMP10 | GCC CAT CTC CAT CCT CTA TTT | 3 | AAG CCA TAG GAC TCT TCT TCT ATC | 4 |
| COLIA1 | CAA TGC TGC CCT TTC TGC TCC TTT | 5 | CAC TTG GGT GTT TGA GCA TTG CCT | 6 |
| COL3A1 | GAA TTT GGT GTG GAC GTT GG | 7 | CTT GCA CTG GTT GAC AAG ATT AG | 8 |
| CDH11 | ACC ACT CAT TGT CTT TGA GGA A | 9 | ATT CTG GAG GGT GGC AAT ATC | 10 |
| CD36 | GGT GAT GAG AAG GCA AAC A | 11 | GGTGATGAGAAGGCAAACA | 12 |
| CTNT | TTC ACC AAA GAT CTG CTC CTC GCT | 13 | TTA TTA CTG GTG TGG AGT GGG TGT GG | 14 |
| DLL4 | CCT GCA TTG TGA ACA CAG CAC CTT | 15 | ACC TGT CCA CTT TCT TCT CGC AGT | 16 |
| ETV2 | CCG GGC ATG AAT TAC GAG AA | 17 | CGA AGC GGT ACG TGT ACT TT | 18 |
| FABP4 | CGT CAC TTC CAC GAG AGT TTA T | 19 | TCC CAC AGA ATG TTG TAG AGT TC | 20 |
| GATA4 | CGA ATG ACG GCA TCT GTT TGC CAT | 21 | ATT TGG TAT TAG GGA TGC AGG GCG | 22 |
| GATA5 | ACC AAG ATT CCC AGT GAA GCA CCT | 23 | TCC GTC TAT CCA TGT GGG CAA TGA | 24 |
| GJA4 | CTA CCT CGT GGA CTG CTT TG | 25 | GTT AAG CAC CAG GGA GAT GAG | 26 |
| GJA5 | AAT CTT CCT GAC CAC CCT GCA TGT | 27 | CAG CCA CAG CCA GCA TAA AGA CAA | 28 |
| HEY2 | GAG TGA GAG AGT CGT GTT TC | 29 | ACT TCT GTC CCT TTC CTT TC | 30 |
| IRX3 | TCT GCC TTT GTG TGT GTG | 31 | GTG GCA GCA GCT CAT TTA | 32 |
| ISL1 | GAA GGT GGA GCT GCA TTG GTT TGA | 33 | TAA ACC AGC TAC AGG ACA GGC CAA | 34 |
| ITGA2 | GAT GAG ATT GAT GAG ACC ACA GA | 35 | TTC ACG CAA ACA GCA AAC C | 36 |
| JAG1 | TGC TAC AAC CGT GCC AGT GAC TAT | 37 | AGT GGT CTT TCA GGT GTG AGC AGT | 38 |
| MSX2 | CAC GCC CTT TAC CAC ATC C | 39 | AGG TTC AGA GAG CTG GAG AA | 40 |
| MYL2 | TGT CCC TAC CTT GTC TGT TAG CCA | 41 | ATT GGA ACA TGG CCT CTG GAT GGA | 42 |
| NFATC1 | CCT GCT GCC TTA CAC AGT GCA TTT | 43 | TGC GAC TAT GAA CAA GCC TAC GGT | 44 |
| NKX2-5 | TTT GCA TTC ACT CCT GCG GAG ACC TA | 45 | ACT CAT TGC ACG CTG CAT AAT CGC | 46 |
| NOTCH1 | CGG GTC CAC CAG TTT GAA TG | 47 | GTT GTA TTG GTT CGG CAC CAT | 48 |
| NOTCH2 | TTT GGC AAC TAA CGT AGA AAC TCA AC | 49 | TGC CAA GAG CAT GAA TAC AGA GA | 50 |
| NPPA | GGG TCT CTG CTG CAT TTG TGT CAT | 51 | AGA GGC GAG GAA GTC ACC ATC AAA | 52 |
| NPPB | TCC TGC TCT TCT TGC ATC TGG CTT | 53 | TGT AAC CCG GAC GTT TCC AAG T | 54 |
| NPR3 | CGG GAA GAT TCC ATC AGA TCC | 55 | GTC TTT CAA CCC GTC TAT CTC C | 56 |
| NR4A2 | CCC AGA ACT TCG TAC CCT TTG | 57 | TGC TTG GGA GGA GGT CTT A | 58 |
| NRG1 | CCG AAA GCC ACT CTG TAA TC | 59 | CCT GAG GAA GCT GTT ACA TTC | 60 |
| POSTN | CCG AGC CTT GTA TGT ATG TTA TG | 61 | TTT GTA CAC CAA GCA CCT ATT T | 62 |
| PRRX2 | TCG CTG CTC AAG TCC TAC A | 63 | CCA GGA GAG ATA ATC TGG ACT CA | 64 |
| RGS5 | TGG ATT GCC TGT GAG GAT TAC | 65 | TTA GGA GCC TCC GTT TGA ATG | 66 |
| SOX9 | GAA AGA GAG GAC CAA CCA GAA T | 67 | TTG GGT ACG AGT TGC CTT TAG | 68 |
| TBP | TGA GTT GCT CAT ACC GTG CTG CTA | 69 | CCC TCA AAC CAA CTT GTC AAC AGC | 70 |
| TIMP3 | GCG TCT ATG ATG GCA AGA TGT A | 71 | ACC CAG GTG ATA CCG ATA GT | 72 |
| VCAN | TGG AAA GAT GAA ACC TCG TTA T | 73 | TCC TAA GCA CCG GAT AGT T | 74 |
| VIM | AGCATCACGATGACCTTGAATA | 75 | GGCACTTGAAAGCTGTTTCTT | 76 |

### Example 18—Generation of Coronary Endothelial-Like Cells From PSC-Derived Endocardial-Like Cells

Several lineage tracing studies have demonstrated the contribution of endocardial cells to the coronary endothelium of the adult, with these cells being preferentially localized to the septal arteries and inner myocardial walls (see, e.g., Zhang et al., 2016, Circ. Res., 118:1880-93; Sharma et al., 2017, Develop. Cell, 42:655-66; Wu et al., 2021, Cell, 151:1083-96). Upon ablation of the other predominant progenitor pool, the sinus venosus endothelium, the endocardial cells will give rise to most of the coronary endothelial cells in the adult (see, e.g., Sharma et al., 2017, Develop. Cell, 42:655-66; Chen et al., 2014, Develop., 141:4500-12). These observations demonstrate that endocardial cells represent a source of coronary endothelium that can function across the vascular bed.

One of the defining characteristics of the coronary arterial endothelium is its capacity to transport fatty acids (FA) to the adjacent cardiomyocytes (see, e.g., Su et al., 2018, Nature, 559:356-62; Hagberg et al., 2010, Nature, 464:917-21). To achieve this, the coronary endothelial cells upregulate expression of genes that encode proteins required for this process, including CD36 and LDLR, proteins involved in the trafficking of FA from the blood stream into the cell, FABP4, a cytosolic FA binding protein, and APOD, a secreted regulator of FA metabolism.

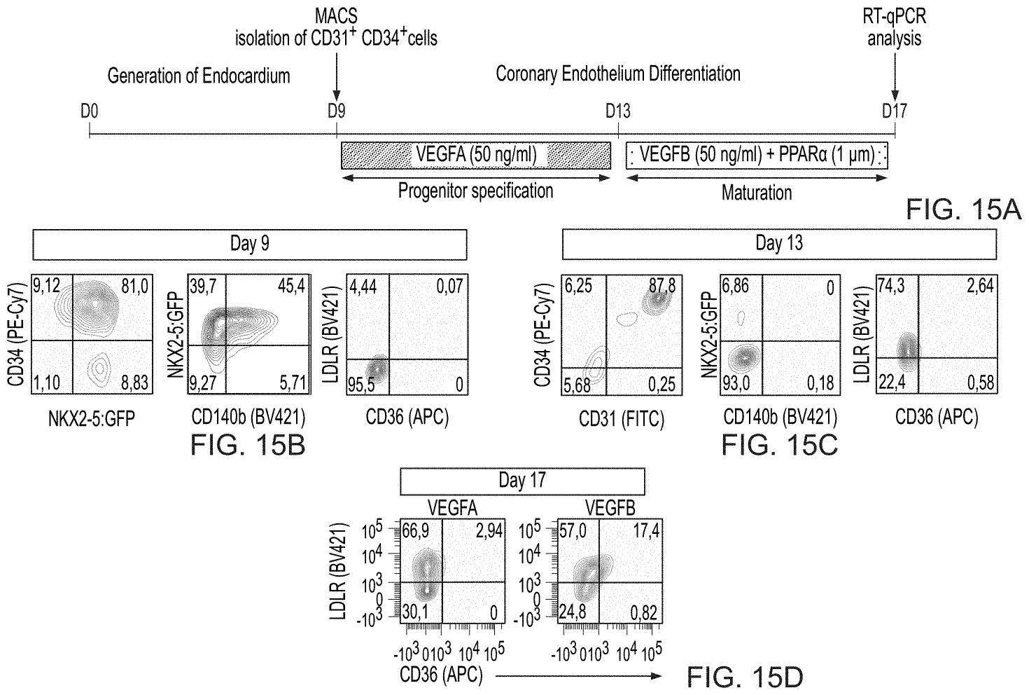

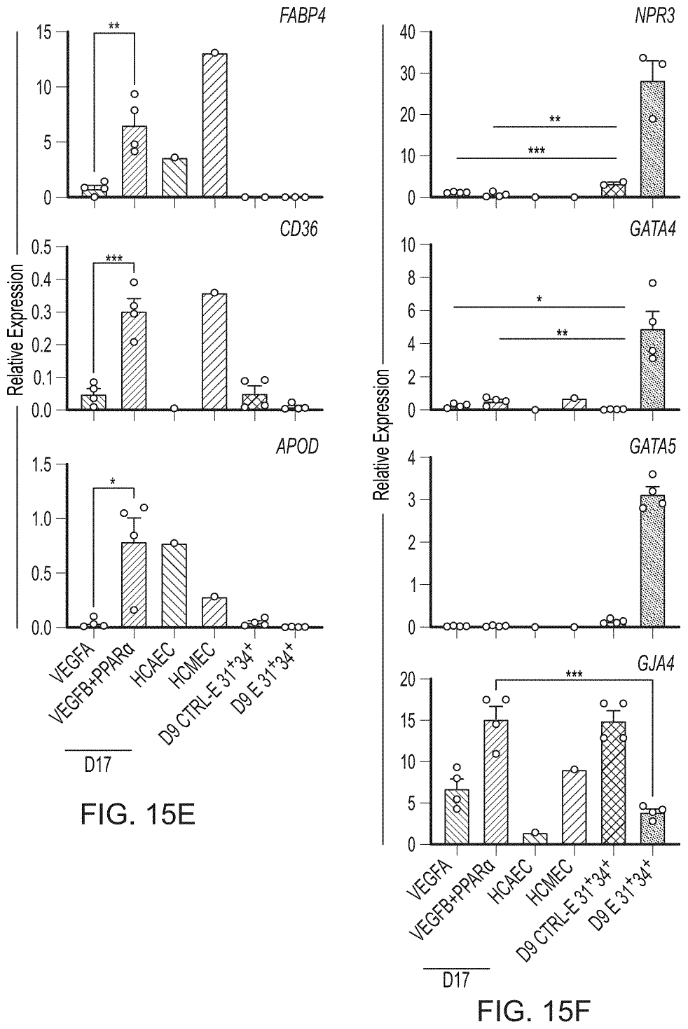

To generate coronary cells in vitro, important signals known to regulate their development from endocardial cells in the developing embryo were recapitulated, using a two-step protocol that involves both specification to immature coronary endothelium and their maturation to functional cells (FIG. 15A) (see, e.g., Wu et al., 2012, Cell, 151:1083-96). NKX2-5+ CD31+ CD34+ hPSC-derived endocardial-like cells were isolated at day 9 using magnetic-activated cell sorting (MACS). A subset of these cells also expressed CD140b (PDGFRb) (FIG. 15B). The endocardial cells do not express CD36 or LDLR, indicating they are not metabolically mature (FIG. 15B).

In the embryo, endocardial cells expand from the subluminal surface of the heart into the developing myocardium through a VEGFA dependent process (see, for example, Wu et al., 2021, Cell, 151:1083-96). This is considered to be the first step in the development of the coronary lineage. To mimic this step, the hPSC-derived endocardial cells were cultured in the presence of VEGFA for 4 days. Treatment with VEGFA promoted the development of a CD34+ CD31+ endothelial cell population that downregulated expression of the endocardial markers NKX2-5 and CD140b and initiated upregulation of LDLR, suggesting specification to the coronary lineage (FIG. 15C). Although the VEGFA-treated cells lost markers of the endocardial lineage and upregulated LDLR, they did not upregulate CD36, a defining marker of the coronary endothelium (FIG. 15C).

In the heart, CD36 expression is regulated by VEGFB secreted from the adjacent cardiomyocytes (Hagberg et al., 2010, Nature, 464:917-21). To recapitulate this stage, the cells were cultured in the presence of VEGFB together with a PPARa agonist, GW7647, a known regulator of fatty acid transport and metabolism (Chanda et al., 2016, FEBS Letters, 590:2364-74). As a control, cells were cultured in VEGFA for the entire culture period. Flow cytometric analyses showed that treatment with the combination of VEGFB and PPARa agonist induced the upregulation of CD36 on a subset of cells expressing LDLR (FIG. 15D). RT-qPCR analysis confirmed these findings and showed an upregulation of CD36 message, together with that of FABP4 and APOD in the VEGFB/PPARa agonist-treated cells (FIG. 15E). In contrast, the treated cells downregulated expression of genes associated with the endocardial fate including NPR3, GATA4 and GATA5 (FIG. 15F). Taken together, these findings demonstrate that the sequential treatment of hPSC-derived endocardial cells with VEGFA and VEGFB/PPARa agonist promotes the development of an endothelial population that displays characteristics of coronary endothelial cells.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of producing a population of endocardial cells, comprising:
providing cardiovascular progenitor cells; and
contacting the cardiovascular progenitor cells with FGF and BMP under appropriate culture conditions,
thereby producing a population of endocardial cells, wherein the population of endocardial cells is phenotypically NKX2-5+ and CD31+.

2. The method of claim 1, wherein the cardiovascular progenitor cells are pluripotent stem cell-derived cardiovascular progenitor cells.

3. The method of claim 1, wherein the cardiovascular progenitor cells are cardiovascular mesoderm cells.

4. (canceled)

5. The method of claim 1, wherein the BMP is selected from BMP10 and BMP4.

6. (canceled)

7. The method of claim 1, wherein the appropriate culture conditions comprises the absence of VEGF or the absence of a Wnt inhibitor.

8. (canceled)

9. The method of claim 1, wherein the population of endocardial cells is phenotypically GATA4+, GATA5+, NFATC +, NPR3+ and NRG1+ or wherein the population of endocardial cells is phenotypically ENG(CD105)+.

10. (canceled)

11. Endocardial cells made by the method of claim 1.

12. The method of claim 1, further comprising:
culturing PDGFRb+ cells from the population of endocardial cells in the presence of bFGF, BMP2 and TGFbeta2,
thereby producing valvular interstitial-like cells (VICs), wherein VICs are phenotypically SOX9+, MSX2+, VIM+, VCAN+, COL1A1+, COL3A1+, POSTN+, CDH11+, NR4A2+, PRRX2+, TIMP3+, RGS5+ and ITGA2+.

13. Valvular interstitial-like cells (VICs) made by the method of claim 12.

14. A method of producing coronary endothelial-like cells, comprising:
contacting the endocardial cells of claim 11 with a) VEGFA followed by b) VEGFB,
thereby producing coronary endothelial-like cells.

15. The method of claim 1, further comprising:
culturing the population of endocardial cells in the presence of ventricular cardiomyocytes, wherein the ventricular cardiomyoctyes are phenotypically MYL2(MLC2V)+ and CTNT+,
thereby generating trabecular myocardial cells, wherein the trabecular myocardial cells are phenotypically BMP10+, NPPA+, NPPB+, IRX3+, GJA5+, MYL2+, and CTNT+.

16. The method of claim 15, wherein the culturing is in the presence of VEGFA.

17. Trabecular ventricular cardiomyocytes made by the method of claim 15, wherein the trabecular ventricular cardiomyocytes are phenotypically BMP10+, NPPA+, NPPB+, IRX3+, GJA5+, MYL2+, and CTNT+.

18. A method of replenishing coronary vasculature in myocardium, comprising:
delivering the endocardial cells of claim 11 to heart tissue,
thereby replenishing the coronary vasculature.

19. (canceled)

20. A method of improving cardiomyocyte grafts by replenishing the myocardium, comprising:
delivering ventricular cardiomyocytes in conjunction with the endocardial cells of claim 11,
thereby replenishing the myocardium.

21-23. (canceled)

24. A method of producing valvular interstitial-like cells, comprising:
contacting the endocardial cells of claim 11 with BMP2/4 and TGFbeta2 under appropriate culture conditions, or
contacting the endocardial cells of claim 11 with a) CHIR99021, SB-431542, bFGF and BMP2/4 followed by b) bFGF, BMP2/4 and TGFbeta2,
thereby producing valvular interstitial-like cells.

25. The method of claim 24, wherein the valvular interstitial-like cells (VICs) are phenotypically SOX9+, MSX2+, VIM+, VCAN+, COL1A1+, COL3A1+, POSTN+, CDH11+, NR4A2+, PRRX2+, TIMP3+, RGS5+ and ITGA2+.

26. A method of producing trabecular ventricular cardiomyocytes, comprising:
contacting ventricular cardiomyocytes with the endocardial cells of claim 11 or exogenous neuregulin (NRG1) under appropriate culture conditions,
thereby producing trabecular ventricular cardiomyocytes.

27. (canceled)

28. A method of inducing the production of neuregulin in a cell, comprising:
contacting the endocardial cells of claim 11 with ventricular cardiomyocytes under appropriate conditions.

29. A biological valve comprising the endocardial cells of claim 11.

30. A method of replenishing coronary vasculature in myocardium, comprising:
delivering the coronary endothelial cells made by the method of claim 14 to heart
thereby replenishing the coronary vasculature.

31. A method of improving cardiomyocyte grafts by replenishing the myocardium, comprising:
delivering ventricular cardiomyocytes in conjunction with the coronary endothelial cells made by the method of claim 14,
thereby replenishing the myocardium.

32. A biological valve comprising the valvular interstitial-like cells of claim 13.
